(19) United States
(12) Patent Application Publication
(10) Pub. No.: US 2021/0069176 A1
(43) Pub. Date: Mar. 11, 2021

(54) Treating Autoimmune Disorders with Chloroquine and/or Hydroxychloroquine

(71) Applicants: Frank Anthony Spallitta, Denver, CO (US); Attillaps Holdings, Denver, CO (US)

(72) Inventor: Frank Anthony SPALLITTA, Denver, CO (US)

(21) Appl. No.: 16/959,929

(22) PCT Filed: Jan. 4, 2019
(86) PCT No.: PCT/US2019/012308

§ 371 Date: Jul. 2, 2020

Related U.S. Application Data
(60) Provisional application No. 62/614,073, filed on Jan. 5, 2018.
(60) Provisional application No. 62/614,078, filed on Jan. 5, 2018.
(60) Provisional application No. 62/614,087, filed on Jan. 5, 2018.

(51) Int. Cl. A61K 31/47; A61K 9/00; A61K 9/06; A61K 47/38; A61K 47/02; A61K 47/12; A61K 9/20; A61K 47/22

(52) U.S. Cl. CPC A61K 31/47; A61K 9/0019; A61K 9/0014; A61K 9/0053; A61K 9/06; A61K 47/22; A61K 47/02; A61K 47/12; A61K 9/2009; A61K 9/2054; A61K 9/2059; A61K 47/38

(57) ABSTRACT

Provided herein are treatments of autoimmune conditions by the topical, oral or intravenous use of an active agent, specifically including chloroquine and/or hydroxychloroquine, in an amount effective to reduce or eliminate mites. By effectively reducing or eliminating the population of Demodex mites in affected areas and areas where Demodex mites may exist, this treatment achieves a more complete remission of clinical signs and symptoms of the autoimmune afflictions than previously described methods. Methods are useful for treating autoimmune diseases.

### CROSS-REFERENCE TO RELATED APPLICATIONS

This application claims the benefit of priority to U.S. Provisional Patent Application Nos. 62/614,073 (“Treating Autoimmune Disorders with Acetylcholinesterase Inhibitors”: 140-17P), 62/614,078 (“Treating Autoimmune Disorders with Ivermectin”: 141-17P), 62/614,087 (“Treating Autoimmune Disorders with Chloroquine and/or Hydroxychloroquine”: 3-18P US) each filed on Jan. 5, 2018, and each specifically incorporated by reference herein to the extent not inconsistent herewith.

### BACKGROUND OF INVENTION

Provided herein are methods for treatment of various autoimmune diseases, including in humans, employing topically applied, orally dosed or intravenously dosed active agents such as (1) acetylcholinesterase inhibitors and/or carbamates, such as ethyl carbamates, organophosphates; (2) avermectins such as ivermectin; and/or (3) chloroquine and/or hydroxychloroquine, to inactivate certain organisms associated with the autoimmune disease. For example, by reducing or eliminating Demodex organisms from the patient, including infected areas, the methods reduce clinical signs or symptoms of the autoimmune disease which are primarily due to inflammatory and/or immune responses of the body to the Demodex organism and/or pathogens that are carried by the Demodex organism.

Although hypothesized as a somewhat benign human ectoparasite, Demodex folliculorum is implicated as playing a causative role in the autoimmune diseases lupus erythematosus, rheumatoid arthritis, juvenile idiopathic arthritis, systemic lupus, systemic sclerosis, juvenile and adult dermatomyositis, Sjögren's syndrome, and for the treatment of porphyria cutanea tarda. No commercially viable pharmacological solutions are available for treating Demodex brevis and Demodex folliculorum in autoimmune disease. Reaction to the presence or metabolic activity of Demodex mites in the human epidermis has been described previously in dermatological conditions such as rosacea and acne vulgaris and the ophthalmological condition meibomian gland dysfunction which is considered to be the leading cause of dry eye disease. A parasitic component of all autoimmune diseases, especially those having a dermatological component, where evidence of use of the drug hydroxychloroquine and/or chloroquine as an effective intervention is believed to have an anti-parasitic component, including one that acts on Demodex in the disease etiology. Compounds, including the organophosphates described in PCT Pub. No. WO2015/017328 and US Pub. No. 2015/0086596; and acetylcholinesterase inhibitors described in PCT Pub. No. WO 2015/195928, US Pub. No. 2017/0135978, that act on the mites may be effective in managing these misunderstood and challenging diseases. See also, Al-Bari, Md Abdul Alim. “Chloroquine analogues in drug discovery: new directions of uses, mechanisms of actions and toxic manifestations from malaria to multifarious diseases.” Journal of Antimicrobial Chemotherapy 70.6 (2015): 1608-1621; and Ben-Zvi, Ilan, et al. “Hydroxychloroquine: from malaria to autoimmunity.” Clinical reviews in allergy & immunology 42.2 (2012): 145-153.

Metrifonate/dichlorvos is a potent irreversible acetylcholinesterase inhibitor that has been used since the 1960's to treat the parasitic infection schistosomiasis (bilharzia). Metrifonate/dichlorvos has been utilized to treat the skin condition rosacea by targeting Demodex folliculorum and Demodex brevis mite in the human epidermis. Therefore, repositioning acetylcholinesterase inhibiting compounds to treat autoimmune diseases where demodectic involvement is suspected provides a new form of treatment for patients suffering from autoimmune disease, including systemic lupus erythematosus, rheumatoid arthritis, juvenile idiopathic arthritis, systemic lupus, systemic sclerosis, juvenile and adult dermatomyositis, Sjögren's syndrome, and porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis.

The etiology of the diseases systemic lupus erythematosus, rheumatoid arthritis, juvenile idiopathic arthritis, systemic lupus, systemic sclerosis, juvenile and adult dermatomyositis, Sjögren's syndrome, and porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis are not well understood. Autoimmune diseases are defined as diseases in which the body produces antibodies that attack its own tissues, leading to the deterioration and in some cases to the destruction of such tissue. The immune system in some people may actually be upregulated by the presence of the ubiquitous human mite Demodex or a pathogen carried on or inside the mite.

Many rosacea flare triggers overlap with autoimmune flare triggers especially lupus (SLE). Lupus rash, much like the rash seen in rosacea patients, tends to occur in the T and U zones of the face. In lupus the rash is referred to as a malar rash or butterfly rash. Antibiotics that treat rosacea are capable of inducing lupus, especially tetracyclines. The antibiotics may act on the mite and that, in turn, causes the medication-induced lupus. The most effective current way of treating medication-induced rosacea is to discontinue the medication that initiated the reaction. It should also be noted that tetracycline antibiotics are used to treat acne, rosacea, and meibomian gland dysfunction. Whether the antibiotics act as an anti-microbial agent and kill the Demodex or act on the mites gut bacteria has yet to be fully elucidated. We are able to show in our in vitro assays that doxycycline does have activity against the mite. (FIG. 3).

The anti-malarial drugs hydroxychloroquine and chloroquine treat autoimmune disease by acting as an anti-parasitic agent against Demodex. The drugs that treat dry eye where Demodex is now suspected to be playing a causative role are treated with the same agents that are being used to treat many of these autoimmune diseases. Cyclosporine and Lifitegrast function by down-regulating the T cell or T lymphocytes of the patient. The immunosuppressant Tacrolimus has also been reported to treat dry eye. These immunosuppressants down-regulate the host's immune response to the Demodex mites in many different diseases that could be redefined as severe allergic reactions or inflammatory response to the Demodex mites or pathogens harbored by the mites.

Demodex mites have been found in the lymphatic glands of dogs. Demodex canis could be used as a surrogate model to better understand Demodex brevis and Demodex folliculorum in humans. It should also be noted that dogs suffer from lupus, referred to as canine discoid lupus erythematosus, which may have an underlying causative effect related to Demodex canis.

Intense pulsed light (IPL) has been used to treat rosacea and dry eye. Demodex has been implicated as playing a causative role in both diseases. IPL treatment has been proven to kill Demodex mites. See Prieto V, et al. in “Effects of intense pulsed light on sun-damaged human skin, routine, and ultrastructural analysis.” (2002); Timothy Kim “Intense pulsed light eradicates Demodex mites.” Skin Allergy News (2002). In 2000 Dr. J L Levy described treating the chronic facial erythema of systemic lupus erythematosus in “Intense pulsed light treatment for chronic facial erythema of systemic lupus erythematosus: a case report.” In the report a 33-year-old woman, who had been diagnosed previously with systemic lupus erythematosus, presented with chronic erythema and rosacea of the face. The patient suffered from flushing and burning of the facial skin and sought prior treatment with anti-malarial drugs. After various treatment options were discussed with the patient, she agreed to undergo intense pulsed light therapy. Improvement was noted after the first session and 75% clearance was observed at 1 month after a second session. There were no adverse effects associated with the treatment. One year later it was observed that the results of the two treatments had been maintained. Photosensitivity is one of the most widespread symptoms of lupus UV rays either from the sun or from artificial light and is the most common reported lupus flare trigger. Using IPL in a lupus patient is very counterintuitive. The resulting clearance in the case report aligns with a mechanism related to removal of the Demodex mite from the patient's facial skin.

No current treatments for these autoimmune diseases provide substantial efficacy. Current treatments focus on improving quality of life through controlling symptoms and minimizing flare-ups. Treatment management includes lifestyle management and medications, such as anti-inflammatories, steroids and immunosuppressants. Other treatments include acetaminophen or nonsteroidal anti-inflammatory drugs (NSAIDs), sometimes in combination with antimalarial drugs, are sometimes used. Acetaminophen and NSAIDS are often enough to reduce symptoms. Antimalarial drugs include those such as hydroxychloroquine (Plaquenil) and chloroquine. Low-dose corticosteroids and/or corticosteroid creams or ointments are used. Corticosteroids, include those such as prednisone. Immunosuppressive medicines, such as azathioprine, belimumab, cyclophosphamide, methotrexate, or mycophenolate mofetil may be used. Corticosteroids are the first-line treatment for muscle involvement in dermatomyositis, but skin lesions often flare by reduction or discontinuation. There is a high unmet need for new therapeutic strategies focusing on skin involvement in systemic autoimmune diseases, including of mites.

U.S. Pat. Pub. 2013/0095051 filed Dec. 6, 2012 describes a method of treating rosacea using avermectin/metronidazole in a topical application. U.S. Pat. No. 5,952,372 describes a method for treating rosacea using ivermectin orally or topically in order to reduce and eliminate the parasite Demodex folliculorum present on the skin of patients.

Ivermectin belongs to the avermectin family, a group of macrocyclic lactones produced by the bacterium Streptomyces avermitilis. The avermectins especially include ivermectin, invermectin, avermectin, abamectin, doramectin, eprinomectin and selamectin. Ivermectin is known for its antiparasitic and anthelmintic properties. The antiparasitic activity is thought to be due to the opening of a chlorine channel in the membrane of the neurons of the parasite under the effect of an increased release of the neuromediator GABA (gammaaminobutyric acid), inducing neuromuscular paralysis that may lead to the death of certain parasites. Ivermectin also interacts with other chlorine channels, especially those dependent on the neuromediator GABA (gammaaminobutyric acid).

Ivermectin is conventionally administered in the dermatological treatment of endoparasitic manifestations such as onchocerciasis and myiasis. U.S. Pat. No. 6,133,310 describes the use of ivermectin in the treatment of rosacea in order to reduce and eliminate the parasite Demodex folliculorum present on the skin of patients. U.S. Pat. No. 6,133,310 describes the use of ivermectin in the treatment of rosacea in order to reduce and eliminate the parasite Demodex folliculorum present on the skin of patients.

Chloroquine and/or hydroxychloroquine (Plaquenil) is human-approved with respect to a number of diseases and conditions, including for malaria.

### SUMMARY OF THE INVENTION

Provided herein are treatment methods that alleviate, abrogate, or otherwise reduce or stop any one or more of the above clinical symptoms by administering or applying an active agent. As used herein, an active agent includes: (1) acetylcholinesterase inhibitors, including a carbamate, a naturally occurring acetylcholinesterase inhibitor, an ethyl carbamate, and/or an organophosphate compound; (2) chloroquine and/or hydroxychloroquine; and/or (3) an avermectin, such as ivermectin.

Aspects of the invention described herein involve treating autoimmune disease by the topical, oral or intravenous administration of an active agent, such as chloroquine and/or hydroxychloroquine; a compound of the avermectin family, such as ivermectin; one or more than one organophosphate compounds, acetylcholinesterase inhibitors including a carbamate, an ethyl carbamate, or a naturally occurring acetylcholinesterase inhibitor. By effectively reducing or eliminating the population of mites in affected areas and areas where mites may exist, this treatment achieves a more complete remission of clinical signs and symptoms of the immune or inflammatory afflictions than any previously described method. Aspects of the invention may be useful for treating a range of autoimmune diseases, such as any one or more of the autoimmune disease systemic lupus erythematosus, rheumatoid arthritis, juvenile idiopathic arthritis, systemic lupus, systemic sclerosis, juvenile and adult dermatomyositis, Sjögren's syndrome, and porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis. The methods provided herein are compatible with inactivating a wide range of mites, wherein the mite is at least partially attributed to the autoimmune disease, including the clinical signs and/or symptoms.

Any of the methods described herein may be for inactivating Demodex mites. Any of the methods may be for inactivating one or more of the following mites: Demodex aries, Demodex aurati, Demodex brevis, Demodex bovis, Demodex canis, Demodex caprae, Demodex caballi, Demodex cati, Demodex conicus, Demodex cornei, Demodex criceti, Demodex equi, Demodex folliculorum, Demodex foveolator, Demodex gapperi, Demodex gatoi, Demodex huttereri, Demodex injai, Demodex leucogasteri, Demodex microti, Demodex ovis, Demodex phyloides, Demodex ponderosus, Demodex vibrissae or Demodex zalophi.

An exemplary method comprises a step of orally-administering or topically-applying to an individual having the autoimmune affliction an active agent such as chloroquine and/or hydroxychloroquine, in a dosage sufficient to inactivate mites, including Demodex brevis and/or Demodex folliculorum, from the body of the individual, resulting in amelioration or cessation of the manifestations of immune and/or inflammatory responses to the mites that cause symptoms and signs of the affliction in the individual. For aspects where an acetylcholinesterase inhibitor is used, an organophosphate may be used, including any of the organophosphates described in WO 2015/017328; the acetylcholinesterase inhibitor, including a carbamate, a naturally occurring acetylcholinesterase inhibitor and/or an ethyl carbamate, may be any of the compounds described in WO 2015/195928, each of which are specifically incorporated by reference for the compounds, formulations, and administration methods described therein.

The chloroquine and/or hydroxychloroquine may be topically applied that is formulated in a carrier lotion, cream, soap, wash, shampoo, gel, impregnated wipe or swab. In an embodiment, for example, the chloroquine and/or hydroxychloroquine has a concentration in the topically applied carrier lotion, cream, soap, wash, shampoo, gel, impregnated wipe or swab selected from the range 0.001 to 5 percent by weight, optionally for some applications selected from the range 0.001 to 5 percent by weight, 0.01 to 1 percent by weight, 0.5% to 2% by weight, or about 1% by weight. In an embodiment, for example, the chloroquine and/or hydroxychloroquine has a concentration in the topically applied carrier lotion, cream, soap, wash, shampoo, gel, impregnated wipe or swab selected from the range 2 to 5 percent by weight, optionally for some applications selected from the range 5 to 10 percent by weight, 10 to 15 percent by weight, 15% to 20% by weight, about 2% by weight, about 5% by weight, about 10% by weight, between 0.001% to 15% by weight, up to 15% by weight, about 15% by weight, up to 20% by weight, or about 20% by weight. In an embodiment, for example, the chloroquine and/or hydroxychloroquine in the topically applied carrier lotion, cream, soap, wash, shampoo, gel, impregnated wipe or swab is provided in a lowest concentration effective for killing the Demodex brevis mites, Demodex folliculorum mites or both.

Because these autoimmune diseases that show dermatological symptoms also cause more systemic issues in lymph nodes like sarcoidosis and lymphadenopathy in an exemplary embodiment, the active agent that is formulated is recommended to be delivered through oral-administration or intravenous administration.

Optionally, a concentration of the active agent in the topically-applied, orally-administered or intravenously administered drug is about 0.001 to 15 percent by weight or about 0.01 to 1 percent by weight. The concentration of the active agent in the topically-applied, orally-administered or intravenously administered drug may be formulated at the lowest effective concentration effective for killing the Demodex mites. The dosage of active agent in the topically-applied, orally-administered or intravenously administered formulation may be less than about 150 mg/kg of body mass or between about 0.01 mg per kg of body mass and 50 mg/kg of body mass. The dosage of active agent in the topically-applied, orally-administered or intravenously administered drug may be formulated at the lowest dose effective for killing the Demodex mites. Optionally, the topically-applied, orally-administered or intravenously administered active agent is encapsulated inside microliposomes before being formulated into the carrier.

In general, methods of the invention include those where the active agent is applied to hair follicles, skin, eyes, eyelids, eyelashes, or meibomian glands areas, where Demodex are known to exist. The topically-applied, orally-administered or intravenously administered active agent may be further applied to areas of the body not yet known to harbor the mite. For example, the topically-applied active agent may be applied to the hair follicles, skin, eyes, eyelids, eyelashes, or meibomian glands areas of the body where Demodex brevis and/or Demodex folliculorum mites exist. The topically-applied, orally-administered or intravenously administered active agent may be applied or delivered to all areas of the body.

Optionally, methods of the invention further comprise a step of applying the active agent, such as an active agent, to the individual's clothing, linens or both clothing and linens. Such application is useful, for example, for preventing the individual's clothing or linens from being a source of Demodex mites to reintroduce onto the individual's skin. Similarly, methods of the invention optionally further comprise a step of orally-administering or topically-applying the active agent to others having contact with the individual in a dosage sufficient to kill and eliminate Demodex brevis and/or Demodex folliculorum mites from hair follicles and/or skin of the others. For example, in embodiments, the others comprise household members, children, spouses, partners, family members or domestic pets.

The topically-applied active agent, such as active agent, may be applied to the hair follicles, skin, eyes, eyelids, eyelashes, or meibomian glands of the individual. For example, the topically-applied active agent penetrates an outer layer of the skin of the individual, thereby exposing the Demodex brevis and/or Demodex folliculorum mites present below the outer layer of the skin in the eyelid to the active agent. The topically-applied active agent may penetrate to a subdermal region of the eyelid of the individual, thereby exposing the Demodex brevis and/or Demodex folliculorum mites present in and around the meibomian glands to the active agent. Certain formulations of the topical active agent useful with the methods of the invention optionally comprise one or more compositions that increase a permeability of the skin, such as dimethyl sulfoxide (DMSO).

The topically-applied, orally-administered or intravenously administered active agent, such as active agent, may be applied at least once and not more than twice daily for a period of about two to 12 weeks. The topically-applied, orally-administered or intravenously administered active agent may be applied to the affected areas and/or to non-affected areas during a first application period, thereby filling and eliminating adult Demodex brevis and/or Demodex folliculorum mites from the individual. The topically-applied, orally-administered or intravenously administered active agent may be further applied to the affected areas and/or to non-affected areas during a second application period, thereby filling and eliminating from the individual Demodex brevis and/or Demodex folliculorum mites that have matured from a larval form and/or an egg form present on and/or in the skin during the first application period. The topically-applied, orally-administered or intravenously administered active agent may be further applied to the affected areas and/or to non-affected areas during a third application period, thereby filling and eliminating from the individual Demodex brevis and/or Demodex folliculorum mites that have matured from a larval form and/or an egg form present on and/or in the individual during the first application period and/or the second application period.

Optionally, the first application period and the second application period are separated by at least five but no more than ten days. Optionally, the first application period and the second application period are separated by at least seven days. In an exemplary embodiment, the first application period and the second application period are separated by a time sufficient to allow the larva form to mature into an adult form and/or to allow the egg form to mature into the adult form.

Optionally, the second application period and the third application period are separated by at least five but no more than ten days. Optionally, the second application period and the third application period are separated by at least seven days. In an exemplary embodiment, the second application period and the third application period are separated by a time sufficient to allow the larva form to mature into an adult form and/or to allow the egg form to mature into the adult form.

In exemplary embodiments, the active agent is topically-applied, orally-administered or intravenously administered in a continued intermittent regime sufficient for prophylactic control of Demodex mite population in the individual.

Optionally, the orally-administered active agent is administered as a daily dose of 10 mg per kg of body mass. Optionally, the orally-administered active agent is administered as a daily dose of 7.5 mg per kg of body mass. Optionally, the orally-administered active agent is administered as a three times per day dose of 5 mg per kg of body mass. Optionally, the orally-administered active agent is repeated about two to four times with spacing of three to seven days between them.

The dosing for the various administration routes may be selected so as to ensure there is sufficient demodex inactivation, including a portion of or substantially all Demodex mites. In this context, “a portion of” refers to greater than 25% and “substantially all” refers to at least 85%, at least 90%, at least 95%, at least 99.5%, or about 99.9% inactivation of total number of mites present.

In various embodiments, the elimination of the Demodex brevis and/or Demodex folliculorum mites from the individual results in a reduction in population of one or more pathogens in the individual. For example, the immune and/or inflammatory responses to the mites may result from a presence of one or more bacteria associated with the mites in the individual. The one or more bacteria may comprise one or more bacteria from the genus staphylococcus or from the genus bacillus. For example, in one embodiment, the one or more bacteria comprise bacillus oleronius bacteria. In one embodiment, for example, the one or more bacteria comprise Staphylococcus epidermidis bacteria. Optionally, the one or more bacteria or other pathogens are present in a digestive system of the Demodex brevis and/or Demodex folliculorum mites.

Provided herein are methods for treating an autoimmune disease comprising a step of topically-applying to an individual having the immune or inflammatory affliction an active ingredient in a dosage sufficient to kill and eliminate Demodex brevis and/or Demodex folliculorum mites from the individual, resulting in cessation of the manifestations of immune and/or inflammatory responses to the mites that cause symptoms and signs of the affliction in the individual. As described, the active agent may be one or more of: (1) acetylcholinesterase inhibitors, including a carbamate, a naturally occurring acetylcholinesterase inhibitor, an ethyl carbamate, and/or an organophosphate compound; (2) chloroquine and/or hydroxychloroquine; (3) an avermectin, such as ivermectin.

The topically-applied, orally-administered or intravenously administered active agent may be delivered to the individual eliminating the Demodex brevis and/or Demodex folliculorum mites from the individual. Again, methods of the invention are useful, for example, for treating autoimmune conditions including: systemic lupus erythematosus, rheumatoid arthritis, juvenile idiopathic arthritis, systemic lupus, systemic sclerosis, juvenile and adult dermatomyositis, Sjögren's syndrome, and porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis. In an aspect, the condition is caused by, exacerbated by or otherwise comorbid with an infestation of Demodex mites, including a pathogen carried by the Demodex mites.

The acetylcholinesterase inhibitor may be a reversible inhibitor. Compounds that are reversible competitive or noncompetitive inhibitors of cholinesterase include those having therapeutic uses, including: Carbamates, Physostigmin, Neostigmine, Pyridostigmine, Ambenonium, Demecarium, Rivastigmine, Phenanthrene derivatives, Galantamine, Caffeine—noncompetitive (also an Adenosine receptor antagonist)[13][14], Piperidines, Donepezil, Tacrine, also known as tetrahydroaminoacridine (THA′), Edrophonium, Huperzine A[15][16], Ladostigil, Ungeremine[17], and Lactucopicrin.

The acetylcholinesterase inhibitor may be a quasi-reversible inhibitor. Compounds which function as quasi-irreversible inhibitors of cholinesterase tend to have use as pesticides. These include organophosphates and carbamates. Examples of organophosphates include: Echothiophate, Diisopropyl fluorophosphates, Cadusafos, Chlorpyrifos, Dichlorvos, Dimethoate, Metrifonate (irreversible), Malathion and Parathion. Examples of carbamates include: Aldicarb; Bendiocarb; Bufencarb; Carbaryl; Carbendazim; Carbetamide; Carbofuran Carbosulfan Chlorbufam; Chloropropham, Ethiofencarb; Formetanate; Methiocarb; Methomyl Oxamyl Phenmedipham, Pinmicarb; Pirimicarb; Propamocarb; Propham, Propoxur; Huperzine A; Galantamine; Onchidal Coumarins.

The acetylcholinesterase inhibitor may correspond to a compound currently used in medicine, including those having an established safety profile in humans. Examples include: Aricept; Aricept ODT; Cognex; donepezil; Exelon; galantamine; Namzaric; Razadyne; rivastigmine; tacrine; phospholine; neostigmine; parathion malathion; dyflos; physostigmine; endrophonium; pyridostigmine; ecothiapate.

The acetylcholinesterase inhibitor may correspond to an organophosphate selected from one or more of acephate, azamethiphos, azinphos ethyl, azinphos methyl, bromophos, bromophos ethyl, cadusofos, carbophenythion, chlormephos, chlorphoxim, chlorpyrifos, chlorpyrifos-methyl, chlorthiophos, chlorvinophos, croumaphos, crotoxyphos, crufomate, cyanofenphos, cyanophos, demephron-O, demephron-S, demeton-O, demeton-S, demeton-S-methyl, demeton-S-methylsulphon, dialifos, diazinon, dichlofenthion, dichlorvos, dicrotophos, dimefphox, dimethoate, dioxabenzophos, dioxathion, disulfoton, ditalmifos, edifenphos, EPBP, EPN, ESP, ethion, ethopropos, etrimfos, famphur, fenamiphos, fenchlorphos, fenitrothion, fensulfothion, fenthion, fenofos, formothion, fosmethilan, heptenophos, isazofos, isofenphos, isothioate, isoxathion, jodfenphos, leptophos, metrifonate, malathion, menazon, mephosfolan, methacrifos, methamidophos, methidathion, mevinphos, monocrotophos, naled, omethoate, oxydemeton-methyl, parathion, parathion-methyl, phenthoate, phorate, phosalone, phosmet, phosphamidon, phosphamidon amide, phospholan, phoxim, pirimiphos-ethyl, pirimiphos-methyl, profenofos, propaphos, propetamphos, prothiofos, prothoate, pyraclofos, pyridaphenthion, quinlphos, schradan, sulfotep, sulprofos, temephos, TEPP, terbufos, tetrachlorvinphos, thiometon, thionazin, triazophos, trichlorfon, vamidothion, a prodrug of these and a pharmaceutically acceptable salt or ester of these. The organophosphate may be dichlorvos or a prodrug or pharmaceutically acceptable salt or ester thereof. The organophosphate may be metrifonate or a prodrug or pharmaceutically acceptable salt or ester thereof.

Also provided are methods of making a pharmaceutically effective active agent with any of the active ingredients described herein that are capable of inactivating demodex organisms (e.g., mites).

### Statements Regarding Chemical Compounds and Nomenclature

In an embodiment, a composition or compound used with the methods of the invention is isolated or purified. In an embodiment, an isolated or purified compound is at least partially isolated or purified as would be understood in the art. In an embodiment, the composition or compound of the invention has a chemical purity of 95%, optionally for some applications 99%, optionally for some applications 99.9%, optionally for some applications 99.99%, and optionally for some applications 99.999% pure.

Many of the compounds used in the methods of the invention contain one or more ionizable groups. Ionizable groups include groups from which a proton can be removed (e.g., —COOH) or added (e.g., amines) and groups which can be quaternized (e.g., amines). All possible ionic forms of such molecules and salts thereof are intended to be included individually in the disclosure herein. With regard to salts of the compounds herein, one of ordinary skill in the art can select from among a wide variety of available counterions that are appropriate for preparation of salts of this invention for a given application. In specific applications, the selection of a given anion or cation for preparation of a salt can result in increased or decreased solubility of that salt.

The term “organophosphate” refers generally to compounds having at least one organophosphate group, or a prodrug thereof, and includes any of the compounds described in U.S. Pat. Pub. No. 2015/0086596 (“Organophosphates for Treating Afflictions of the Skin”), which is specifically incorporated by reference herein for the organophosphates described therein. In some embodiments, for example, the organophosphate is an ester of phosphoric acid (H₃PO₄). In some embodiments, for example, the organophosphate is a halogenated ester of phosphoric acid (H₃PO₄), such as a chlorinated or brominated ester of phosphoric acid (H₃PO₄). In some embodiments, for example, the organophosphate is represented by the structure PO₄R′R″R′″, where each of R′, R″ and R′″ is independently hydrogen or an organic group or substituted organic group, and wherein at least one of R′, R″ and R′″ is not hydrogen. In an embodiment, for example, each of R′, R″ and R′″ are independently hydrogen or a substituted or nonsubstituted alkyl group, alkenyl group, aryl group, heteroaryl group, arylalkyl group, acyl group, alkynyl group, alkoxycarbonyl, halo group, amino group or any combination of these. In an embodiment, for example, at least one of R′, R″ and R′″ is a dichlorovinyl group, such as a group having the formula CCl₂═CH—, and optionally the other(s) of R′, R″ and R′″ are independently hydrogen or a C₁-C₅ alkyl group, and optionally for some application a methyl group. In an embodiment, the organophosphate is 2,2-dichlorovinyl dimethyl phosphate, or a derivative or prodrug thereof. In an embodiment, the organophosphate is isolated or purified, for example, prior to formulation and/or administration.

Organophosphates useful in the methods and compositions of the invention include, but are not limited to acephate, azamethiphos, azinphos ethyl, azinphos methyl, bromophos, bromophos ethyl, cadusofos, carbophenythion, chlormephos, chlorphoxim, chlorpyrifos, chlorpyrifos-methyl, chlorthiophos, chlorvinophos, croumaphos, crotoxyphos, crufomate, cyanofenphos, cyanophos, demephron-O, demephron-S, demeton-O, demeton-S, demeton-S-methyl, demeton-S-methylsulphon, dialifos, diazinon, dichlofenthion, dichlorvos, dicrotophos, dimefphox, dimethoate, dioxabenzophos, dioxathion, disulfoton, ditalmifos, edifenphos, EPBP, EPN, ESP, ethion, ethopropos, etrimfos, famphur, fenamiphos, fenchlorphos, fenitrothion, fensulfothion, fenthion, fenofos, formothion, fosmethilan, heptenophos, isazofos, isofenphos, isothioate, isoxathion, jodfenphos, leptophos, malathion, menazon, mephosfolan, methacrifos, methamidophos, methidathion, mevinphos, monocrotophos, naled, omethoate, oxydemeton-methyl, parathion, parathion-methyl, phenthoate, phorate, phosalone, phosmet, phosphamidon, phosphamidon amide, phospholan, phoxim, pirimiphos-ethyl, pirimiphos-methyl, profenofos, propaphos, propetamphos, prothiofos, prothoate, pyraclofos, pyridaphenthion, quinlphos, schradan, sulfotep, sulprofos, temephos, TEPP, terbufos, tetrachlorvinphos, thiometon, thionazin, triazophos, trichlorfon, vamidothion, any prodrug of these, any pharmaceutically acceptable salt or ester of these and any combination thereof.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” refers to a compound wherein a hydrogen is replaced by another functional group.

As used herein, the term “halo” refers to a halogen group such as a fluoro (—F), chloro (—Cl), bromo (—Br), iodo (—I) or astato (—At).

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenylgroups can also carry alkyl groups. Cycloalkenylgroups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms.

As to any of the groups described herein which contain one or more substituents, it is understood that such groups do not contain any substitution or substitution patterns which are sterically impractical and/or synthetically non-feasible. In addition, the compounds of this invention include all stereochemical isomers arising from the substitution of these compounds. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —ON;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

The term “carbamate” generally refers to an organic compound derived from carbamic acid (NH₂COOH), such as NR₂R₃COOR₁:

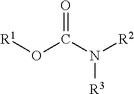

In an aspect, each of the groups R1-R3 are independently selected to correspond to any of the R groups of the chemicals listed herein. In an aspect, any of R1-R3 are hydrogen.

Examples of carbamates for use with the methods described herein include, but are not limited to, neostigmine, rivastigmine, meprobamate, carisoprodol, felbamate, tybamate. Preferred carabamates are those that have been demonstrated to have miticidal or insecticidal capabilities and that can be provided to a mite on or in hair follicles, skin, eyes, eyelids, eyelashes, or meibomian glands at a level sufficient to inactivate or kill the mite without permanently adversely affecting the host patient. The carbamate may be a naturally occurring compound, such as a purified and isolated naturally occurring compound. Alternatively, the carbamate may be a synthetically produced carbamate, as known in the art. Any of the compounds provided herein may be provided in the form of a derivative, prodrug, or a pharmaceutically acceptable salt thereof.

In an aspect, the carbamate is selected from the group consisting of: aldicarb, bendiocarb, bufencarb, carbaryl, carbendazim, carbetamide, carbofuran, carbosulfan, chlorbufam, chloropropham, ethiofencarb, formetanate, methiocarb, methomyl, oxamyl, phenmedipham, pinmicarb, pirimicarb, propamocarb, propham, propoxur, butocarboxim, carbanolate, promacyl, thiocarboxime, thiofanox, benomyl, and metolcarb or a derivative, prodrug or pharmaceutically acceptable salt thereof.

In an aspect, the carbamate is an ethyl carbamate of the form R₁=ethyl. R₂ and R₃ are optionally independently selected as hydrogen.

The compounds used in the methods of this invention can contain one or more chiral centers. Accordingly, this invention is intended to include racemic mixtures, diasteromers, enantiomers, tautomers and mixtures enriched in one or more stereoisomer. The scope of the invention as described and claimed encompasses the racemic forms of the compounds as well as the individual enantiomers and non-racemic mixtures thereof.

Pharmaceutically acceptable salts comprise pharmaceutically-acceptable anions and/or cations. As used herein, the term “pharmaceutically acceptable salt” can refer to acid addition salts or base addition salts of the compounds in the present disclosure. A pharmaceutically acceptable salt is any salt which retains at least a portion of the activity of the parent compound and does not impart significant deleterious or undesirable effect on a subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, -32-cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts may be derived from amino acids, including but not limited to cysteine. Other pharmaceutically acceptable salts may be found, for example, in Stahl et al., Handbook of Pharmaceutical Salts: Properties, Selection, and Use, Wiley-VCH; Verlag Helvetica Chimica Acta, Zurich, 2002. (ISBN 3-906390-26-8). Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

Any of the methods described herein may be used to alleviate in a subject symptoms associated with an autoimmune disease caused by Demodex organisms, the method comprising administering an active agent to the subject having the autoimmune disease in a dosage sufficient to inactivate at least a portion of the Demodex organisms. The active agent may be one or more of an acetylcholinesterase inhibitor, an avermectin such as ivermectin, or chloroquine and/or hydroxychloroquine.

Representative claims of the instant invention include those where the active ingredient is chloroquine and/or hydroxychloroquine, such as any one or more of:
1. A method of treating an individual having an autoimmune disease, the method comprising the step of administering to the individual chloroquine and/or hydroxychloroquine in a dosage sufficient to inactivate at least a portion of Demodex mites in or on the individual, wherein the inactivation results in attenuation or cessation of one or more symptoms associated with inflammatory and/or immune responses to the Demodex mites that causes one or more symptoms associated with the autoimmune disease in the individual.
2. The method of claim 1, wherein substantially all of the Demodex mites are inactivated.
3. The method of claim 1, wherein the administering is by topical, oral or intravenous administration of the chloroquine and/or hydroxychloroquine.
4. The method of claim 1, wherein said chloroquine and/or hydroxychloroquine is a miticide or insecticide.
5. The method of claim 1, wherein the inactivated Demodex mites comprise demodex brevis and/or demodex folliculorum mites from hair follicles, skin, eyes, eyelids, eyelashes, or meibomian glands of the individual.
6. The method of any of claims 1-5, wherein said chloroquine and/or hydroxychloroquine is topically applied and is provided in a formulation to efficiently transport an active ingredient of said chloroquine and/or hydroxychloroquine into the epidermis or a subdermal region of the individual.
7. The method of claim 6, wherein said chloroquine and/or hydroxychloroquine is applied to hair follicles, skin, eyes, eyelids, eyelashes, and/or meibomian glands of the individual.
8. The method of any of claims 1-5, wherein said administering step is by orally-administering or topically-applying, and said inactivation kills at least a portion of said Demodex mites, or renders at least a portion of said Demodex mites unable to reproduce.
9. The method of claim 1, wherein said step of orally-administering or topically-applying said chloroquine and/or hydroxychloroquine kills and eliminates said mites, and optionally said mites are Demodex brevis and/or Demodex folliculorum mites.
10. The method of any of claims 1-9, wherein the autoimmune disease is one or more of lupus erythematosus, rheumatoid arthritis, juvenile idiopathic arthritis, systemic lupus, systemic sclerosis, juvenile dermatomyositis, adult dermatomyositis, Sjögren's syndrome or porphyria cutanea tarda.
11. The method of any of claims 1-9, wherein the autoimmune disease is one or more of systemic lupus erythematosus, rheumatoid arthritis, juvenile idiopathic arthritis, systemic lupus, systemic sclerosis, juvenile and adult dermatomyositis, Sjögren's syndrome, porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis.
12. The method of any of claims 1-9, wherein the autoimmune disease affects the epidermis of the individual.
13. The method of any of claims 1-9, wherein the autoimmune disease affects the epidermis, the lymphatic system, muscles, joint or internal organs of the individual.
14. The method of any of claims 1-13, wherein the chloroquine and/or hydroxychloroquine is topically-applied and is formulated in a carrier lotion, cream, soap, wash, shampoo or gel.
15. The method of claim 14, wherein the chloroquine and/or hydroxychloroquine in the topically-applied lotion, cream, soap, wash, shampoo or gel has a concentration of between 0.001% to 5% by weight.
16. The method of claim 14, wherein the chloroquine and/or hydroxychloroquine in the topically-applied lotion, cream, soap, wash, shampoo or gel has a concentration of between 0.01% to 1% by weight.
17. The method of claim 14, wherein the chloroquine and/or hydroxychloroquine in the topically-applied lotion, cream, soap, wash, shampoo or gel has a concentration that is a lowest effective concentration for killing Demodex mites.
18. The method of claim 14, wherein a dosage of the chloroquine and/or hydroxychloroquine in the topically-applied lotion, cream, soap, wash, shampoo or gel is less than 150 mg/kg of body mass or between 0.001 mg per kg of body mass and 50 mg/kg of body mass.
19. The method of claim 18, wherein a dosage of the chloroquine and/or hydroxychloroquine in the topically-applied lotion, cream, soap, wash, shampoo or gel is a lowest dose effective for killing the Demodex mites.
20. The method of claim 14, wherein the topically-applied chloroquine and/or hydroxychloroquine is encapsulated inside microliposomes before being formulated into the carrier lotion, cream, soap, wash, shampoo or gel.
21. The method of claim 14, wherein the topically-applied chloroquine and/or hydroxychloroquine is applied to hair follicles, skin, eyes, eyelids, eyelashes, or Meibomian Gland areas affected by the ophthalmological affliction.
22. The method of claim 1, wherein the chloroquine and/or hydroxychloroquine is orally or intravenously delivered and is provided at a concentration that is the lowest concentration effective for killing Demodex mites.
23. The method of claim 1, wherein the chloroquine and/or hydroxychloroquine is orally or intravenously delivered with a dose that is less than 150 mg/kg of body mass or between 0.001 mg per kg of body mass and 50 mg/kg of body mass.
24. The method of claim 2, wherein chloroquine and/or hydroxychloroquine is topically-applied to hair follicles, skin, eyes, eyelids, eyelashes, or meibomian gland areas of the body where Demodex brevis and/or Demodex folliculorum mites exist.
25. The method of claim 24, wherein the topically-applied chloroquine and/or hydroxychloroquine is applied to substantially all hair follicles, skin, eyes, eyelids, eyelashes, or meibomian gland areas of the individual.
26. The method of any of claims 1-25 further comprising a step of applying the chloroquine and/or hydroxychloroquine to an individual's clothing, linens or both clothing and linens.
27. The method of any of claims 1-26, further comprising a step of orally-administering or topically-applying the chloroquine and/or hydroxychloroquine to others having contact with the individual in a dosage sufficient to kill and eliminate Demodex brevis and/or Demodex folliculorum mites from hair follicles and/or skin of the others.
28. The method of claim 27, wherein the others are selected from the group consisting of household members, children, spouses, partners, family members and domestic pets.
29. The method of claim 28, wherein the topically-applied chloroquine and/or hydroxychloroquine is applied to the hair follicles and/or skin of the individual.
30. The method of claim 1, wherein the chloroquine and/or hydroxychloroquine is topically applied and penetrates an outer layer of the skin of the individual, thereby exposing the Demodex brevis and/or Demodex folliculorum mites present below the outer layer of the skin to the chloroquine and/or hydroxychloroquine.
31. The method of claim 30, wherein the topically-applied chloroquine and/or hydroxychloroquine penetrates to a subdermal region of the skin of the individual, thereby exposing the Demodex brevis and/or Demodex folliculorum mites present in the subdermal region of the skin to the chloroquine and/or hydroxychloroquine.
32. The method of any of claims 1-32, wherein the chloroquine and/or hydroxychloroquine has mite inactivation activity at any one or more of: hair follicles, skin, eyes, eyelids, eyelashes, or meibomian gland areas by application at least once and not more than twice daily for a period of two to six weeks.
33. The method of claim 32, wherein the chloroquine and/or hydroxychloroquine is applied to the skin or portions thereof, during a first application period, thereby killing and eliminating adult Demodex brevis and/or Demodex folliculorum mites from hair follicles, skin, eyes, eyelids, eyelashes, or meibomian glands.
34. The method of claim 33, wherein the chloroquine and/or hydroxychloroquine is further applied to hair follicles, skin, eyes, eyelids, eyelashes, or meibomian during a second application period, thereby killing and eliminating from the said hair follicles, skin, eyes, eyelids, eyelashes, or meibomian glands Demodex brevis and/or Demodex folliculorum mites that have matured from a larval form and/or an egg form present on and/or in the said hair follicles, skin, eyes, eyelids, eyelashes, or meibomian glands during the first application period.
35. The method of claim 34, wherein the applied chloroquine and/or hydroxychloroquine is further applied to skin areas during a third application period, thereby killing and eliminating from said hair follicles, skin, eyes, eyelids, eyelashes, or meibomian glands Demodex brevis and/or Demodex folliculorum mites that have matured from a larval form and/or an egg form present on and/or in the said hair follicles, skin, eyes, eyelids, eyelashes, or meibomian glands during the first application period and/or the second application period.
36. The method of claim 34 or claim 35, wherein the first application period and the second application period are separated by at least five but no more than ten days.
37. The method of any of claims 34-35, wherein the first application period and the second application period are separated by at least seven days.
38. The method of any of claims 34-35, wherein the first application period and the second application period are separated by a time sufficient to allow the larva form to mature into an adult form and/or to allow the egg form to mature into the adult form.
39. The method of any of claims 35-38, wherein the second application period and the third application period are separated by at least five but no more than ten days.
40. The method of any of claims 35-38, wherein the second application period and the third application period are separated by at least seven days.
41. The method of any of claims 35-38, wherein the second application period and the third application period are separated by a time sufficient to allow the larva form to mature into an adult form and/or to allow the egg form to mature into the adult form.
42. The method of claim 1, wherein the chloroquine and/or hydroxychloroquine is intravenously-administered, orally-administered or topically-applied in a continued intermittent regime sufficient for prophylactic control of Demodex mite population in hair follicles, skin, eyes, eyelids, eyelashes, or meibomian glands of the individual.
43. The method of claim 42, wherein the chloroquine and/or hydroxychloroquine is intravenously administered at an chloroquine and/or hydroxychloroquine dose of 150 mg per kg of body mass or less or between 0.01 mg per kg of body mass and 50 mg per kg of body mass.
44. The method of claim 42, wherein the chloroquine and/or hydroxychloroquine is orally administered.
45. The method of claim 44, wherein the orally-administered chloroquine and/or hydroxychloroquine is administered as an oral dose of the chloroquine and/or hydroxychloroquine of a lowest dose effective for killing the Demodex mites.
46. The method of claim 44, wherein the orally-administered chloroquine and/or hydroxychloroquine is administered as a daily dose of 10 mg per kg of body mass.
47. The method of any of claims 44-45, wherein the orally-administered chloroquine and/or hydroxychloroquine is administered as a daily dose of 7.5 mg per kg of body mass.
48. The method of claim 44, wherein the orally-administered chloroquine and/or hydroxychloroquine is administered as a three times per day dose of 5 mg per kg of body mass.
49. The method of claim 44, wherein the orally-administered chloroquine and/or hydroxychloroquine is repeated about two to four times with spacing of three to seven days between them.
50. The method of claim 43, wherein the intravenous-administered chloroquine and/or hydroxychloroquine is administered as an intravenous dose of the chloroquine and/or hydroxychloroquine of a lowest dose effective for killing the Demodex mites.
51. The method of claim 43, wherein the intravenous-administered chloroquine and/or hydroxychloroquine is administered as a daily dose of 10 mg per kg of body mass.
52. The method of claim 43, wherein the intravenous-administered chloroquine and/or hydroxychloroquine is administered as a daily dose of 7.5 mg per kg of body mass.
53. The method of claim 43, wherein the intravenous-administered chloroquine and/or hydroxychloroquine is administered as a three times per day dose of 5 mg per kg of body mass.
54. The method of claim 43, wherein the intravenous-administered chloroquine and/or hydroxychloroquine is repeated about two to four times with spacing of three to seven days between them.
55. The method of any of claims 1-54, wherein the orally-administered or intravenous-administered chloroquine and/or hydroxychloroquine is formulated as a prodrug or pharmaceutically acceptable salt.
56. The method of claim 1, wherein an immune and/or inflammatory pathway response to the mites results from a presence of one or more pathogens associated with the mites in the individual.
57. The method of claim 56, wherein the elimination of the Demodex brevis and/or Demodex folliculorum mites from the individual results in a reduction in population of the one or more pathogens in the individual.
58. The method of claim 56 or claim 57, wherein the one or more pathogens comprise one or more bacteria from the genus staphylococcus or from the genus bacillus.
59. The method of claim 58, wherein the one or more bacteria comprise bacillus oleronius bacteria.
60. The method of claim 58, wherein the one or more bacteria comprise Staphylococcus epidermidis bacteria.
61. The method of any of claims 56-60, wherein the one or more pathogens are present on the outside of the Demodex brevis and/or Demodex folliculorum mites.
62. The method of any of claims 56-61, wherein the one or more pathogens are present inside of the Demodex brevis and/or Demodex folliculorum mites.
63. The method of any of claims 56-62, wherein the one or more bacteria are present in a digestive system of the Demodex brevis and/or Demodex folliculorum mites.
64. A method of treating an autoimmune disease comprising a step of topically, intravenously or orally delivering to an individual having the autoimmune disease an active ingredient comprising an chloroquine and/or hydroxychloroquine in a dosage sufficient to inactivate Demodex mites from the individual, resulting in amelioration or cessation of the manifestations of immune and inflammatory responses to the mites that cause symptoms and signs of the autoimmune disease in the individual, wherein the active ingredient is applied or delivered to areas affected by the autoimmune disease and to areas not affected by the autoimmune disease.
65. The method of claim 64, wherein the Demodex mites are one or more of: Demodex aries, Demodex aurati, Demodex brevis, Demodex bovis, Demodex canis, Demodex caprae, Demodex caballi, Demodex cati, Demodex conicus, Demodex cornei, Demodex criceti, Demodex equi, Demodex folliculorum, Demodex foveolator, Demodex gapperi, Demodex gatoi, Demodex huttereri, Demodex injai, Demodex leucogasteri, Demodex microti, Demodex ovis, Demodex phyloides, Demodex ponderosus, Demodex vibrissae and Demodex zalophi.
66. The method of claim 64, wherein the Demodex mites are one or more of: Demodex brevis or Demodex folliculorum.
67. The method of claim 64, wherein the topically-applied active ingredient is applied to substantially all skin of the individual, thereby killing and eliminating the Demodex brevis and/or Demodex folliculorum mites from all skin of the individual.
68. The method of claim 64, wherein the active ingredient comprises a miticide or insecticide.
69. The method of claim 64, wherein the autoimmune disease comprises one or more of: systemic lupus erythematosus, rheumatoid arthritis, juvenile idiopathic arthritis, systemic lupus, systemic sclerosis, juvenile and adult dermatomyositis, Sjögren's syndrome, porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis.
70. A method of treating an autoimmune affliction comprising a step of orally-administering, intravenously-administering or topically-applying to an individual having the autoimmune affliction chloroquine and/or hydroxychloroquine in a dosage sufficient to inactivate Demodex brevis and/or Demodex folliculorum mites from the individual resulting in amelioration or cessation of the manifestations of allergic and/or inflammatory responses to the mites that cause symptoms and signs of the autoimmune affliction in the individual.
71. The method of claim 70, wherein the mites are selected from the group consisting of: Demodex brevis; Demodex folliculorum; and Demodex brevis and Demodex folliculorum.
72. The method of claim 70, comprising administering chloroquine.
73. The method of claim 70 or 71, comprising administering hydroxychloroquine.
74. A method of alleviating in a subject symptoms associated with an autoimmune disease caused by Demodex organisms, the method comprising administering chloroquine and/or hydroxychloroquine to the subject having the autoimmune disease in a dosage sufficient to inactivate at least a portion of the Demodex organisms.
75. The method of claim 74, further comprising the step of identifying a subject in need of alleviating symptoms associated with the autoimmune disease caused by demodex organisms.
76. The method of claim 74 or 75, further comprising the step of monitoring whether the subject experiences symptom alleviations.
77. The method of any of claims 74-76, wherein the administration is topically, orally, or intravenously providing the chloroquine and/or hydroxychloroquine to the subject.
78. The method of any of claims 74-77, wherein the administration is a topical administration.
79. The method of claim 78, wherein the topical administration comprises applying the chloroquine and/or hydroxychloroquine to the face and hair follicles of the face and head.
80. The method of claim 78, wherein the topical administration comprises applying the chloroquine and/or hydroxychloroquine to substantially the entire body.
81. The method of any of claims 74-80, further comprising the step of sampling demodex levels in the patient after the administering step.
82. The method of claim 82, wherein the sampling step comprises one or more of visualization of a skin surface, swabbing a skin surface, removing hair, a skin surface biopsy using an adhesive; a skin biopsy, or staining to visualize demodex such as by Löffler's alkaline methylene blue staining.
83. The method of claim 14, wherein the chloroquine and/or hydroxychloroquine in the topically-applied lotion, cream, soap, wash, shampoo or gel has a concentration of between 0.001 percent to 15 percent by weight.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 is an illustration of Demodex folliculorum and Demodex brevis mites, including relative sizes and locations in the skin, hair follicles and glands. FIG. 1 is adapted from Murube J. (2015) Demodex hominis. Ocul Surf. 13(3): 181-186.

FIG. 2 illustrates Demodex mites in the eye region, including eye lash and meibomian glands. FIG. 2 is adapted from Crystal D. (2016) Know your enemy: rich snippet on demodex. etCETera. 2(1): 12-17.

FIG. 3 is a table summary of Demodex survival time for various active agents.

FIG. 4 summarizes the activity of 10% chloroquine in water relative to a water control on activity against Demodex mites in an in vitro assay, with an LT₅₀ of 31.6 hours for chloroquine compared to 44.8 hours for the water control, illustrating the inactivation effect of chloroquine on Demodex.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Active agent” is intended to specifically include: (1) acetylcholinesterase inhibitors, including a carbamate, a naturally occurring acetylcholinesterase inhibitor, an ethyl carbamate, and/or an organophosphate compound; (2) chloroquine and/or hydroxychloroquine; and/or (3) an avermectin, such as ivermectin. Accordingly, anywhere where any of compounds (1), (2) or (3) are listed, it is intended that the specified compounds are interchangeable. For example, wherever ivermectin or acetylcholinesterase inhibitor is used, it is intended that any of chloroquine/hydroxychloroquine may be used instead (and vice versa).

“Inactivate” is used broadly herein to refer to the functional ability to decrease the impact of Demodex brevis and/or Demodex folliculorum mites. For example, the inactivation may be by death of the mite. Alternatively, the inactivation may refer to the inability of the mite to reproduce, so that the mite die off occurs as the mites age and die without reproduction. So long as the treatment leads to an adverse effect on the Demodex brevis and/or Demodex folliculorum mites that corresponds to improved clinical outcome, such as symptom improvement, the treatment is considered herein to inactivate Demodex brevis and/or Demodex folliculorum mites.

“Symptoms associated with inflammatory and/or immune responses to the Demodex mites” refers to the symptoms associated with the disease. For example, the symptom may be related to a symptom of the skin, such as a rash, discoloration, swelling or irritation/tenderness. The symptom may be related to other tissue areas that are associated with an inflammatory disease, such as pain, tenderness, swelling of a muscle or joint. Similar effects may be experienced with an internal organ, lymphatic system, and the like. The treatments provided herein attenuate, alleviate or essentially stop one or more of such symptoms, depending on the specific disease condition. This can be as determined by the patient or by a third-party (medical caregiver) observation or test.

“Sampling” refers to determining the level of demodex organisms on a patient. The sampling can provide an indication as to the efficacy of the treatment with respect to inactivation of demodex. The methods provided herein are compatible with a wide range of sampling techniques, including one or more of visualization of a skin surface, swabbing a skin surface, removing hair, a skin surface biopsy using an adhesive; a skin biopsy, or staining to visualize demodex such as by Löffler's alkaline methylene blue staining (see, e.g., Kiuchi “Better detection of Demodex mites by Löffler's alkaline methylene blue staining in patients with blepharitis.” Clinical Ophthalmology 12: 727-731 (Apr. 16, 2018)).

As used herein, “Demodex” includes D. folliculorum and D. brevis mites, including Demodex mites in humans that may contribute to a demodex-induced inflammatory state or condition in humans, including a state having clinically noticeable effects, such as pain, swelling, tissue tenderness and allergic-type reactions.

“Efficiently transported” refers to the ability of the treatment agent to act against mites that are located beneath the skin surface, such as into an epidermal or subdermal region so that the mites are timely inactivated.

“Substantially all” refers to, unless defined in the contrary, at least 90%, at least 95% or at least 99% of the relevant population, so in the context of demodex mites, it refers to inactivation (e.g., killed or eliminated or otherwise unable to propagate) and/or application to hair (number) or skin (surface area).

In the context of application of active ingredient to an individual's body, such as a skin surface, “substantially all” refers to at least 60%, at least 75%, at least 90%, at least 95%, or at least 99%. This is a recognition that it is difficult to apply a material to 100% of the skin surface, and that the methods provided herein have tolerance with respect to the amount of a biological surface the active ingredient is applied, so long as sufficient fraction of demodex mites are inactivated to alleviate symptoms associated with the autoimmune disorder, and to even treat the underlying autoimmune disorder.

Representative autoimmune diseases useful with the methods provided herein include, Lupus erythematosus including: Systemic lupus erythematosus, or SLE, is the most common form of lupus. Discoid lupus erythematosus causes a skin rash that doesn't go away. Subacute cutaneous lupus erythematosus causes skin sores on areas of the body exposed to the sun. Neonatal lupus. rheumatoid arthritis, juvenile idiopathic arthritis, systemic lupus, systemic sclerosis, juvenile and adult dermatomyositis, Sjögren's syndrome, porphyria cutanea tarda, autoimmune hepatitis, collagen vascular disease, polymyositis, mixed connective tissue disease.

The methods provided herein are compatible with a range of diseases implicated by the presence of mites, specifically diseases where a person's immune system mistakenly attacks the person's own body; e.g., autoimmune diseases. For example, autoimmune diseases include those where the immune system results in deleterious attack on the person's joints (rheumatoid arthritis; Sjogren's syndrome), skin (psoriasis/psoriatic arthritis), a specific biological component (multiple sclerosis for myelin sheath of nerve cells; myasthenia gravis that affects nerves that help the brain control the muscles; vasculitis for blood vessels; pernicious anemia for a protein that helps absorption of vitamin B-12, leading to reduced red blood cell count; celiac disease), a specific organ, pancreas (Type 1 diabetes) bowel (inflammatory bowel disease), adrenal glands (Addison's disease), thyroid gland (Graves' disease; Hashimoto's thyroiditis) or whole body (systemic lupus erythematosus).

Accordingly, there is a wide range of symptoms associated with the inflammatory/immune response, depending on the specific autoimmune disease. With this in mind, “attenuation or cessation of one or more symptoms” may refer to at least a patient-detectable reduction in a symptom associated with the disease, including fatigue, achy muscles, swelling and redness, fever, concentration, numbness and tingling in hands and feet, hair loss, skin rashes, scales or plaques, thirst, weight loss, belly pain, bloating, diarrhea, reduction in flare-up length and/or time, increase in remission times and frequency, joint pain or sensitivity, balance issues, heat intolerance, nervousness, increased heart-beat, dry eyes, dry mouth, cold sensitivity. Similarly, the attenuation may be as determined or assessed by a medical caregiver or specialized medical testing, such as glucose level or one or more markers of elevated immune activity, such as by an antinuclear antibody test (ANA), autoantibodies level, inflammation level and reactivity.

The methods provided herein are also compatible with diseases where ANAs (antinuclear antibodies) are found and used as a diagnostic marker of an autoimmune disease. ANAs are found in many disorders, as well as some healthy individuals. These disorders include: systemic lupus erythematosus (SLE), rheumatoid arthritis, Sjögren's syndrome, scleroderma, polymyositis, dermatomyositis, primary biliary cirrhosis, drug induced lupus, autoimmune hepatitis, multiple sclerosis, discoid lupus, thyroid disease, antiphospholipid syndrome, juvenile idiopathic arthritis, psoriatic arthritis, juvenile dermatomyositis, idiopathic thrombocytopaenic purpura, infection and cancer. These antibodies can be subdivided according to their specificity, and each subset has different propensities for specific disorders.

Patients with the following systemic autoimmune diseases may have a positive test for antinuclear antibodies (ANA): Systemic lupus erythematosus (SLE) (see “Patient education: Systemic lupus erythematosus (SLE) (Beyond the Basics)”); Scleroderma; Sjögren's syndrome (see “Patient education: Sjögren's syndrome (Beyond the Basics)”); Mixed connective tissue disease; Drug-induced lupus; Polymyositis/dermatomyositis (see “Patient education: Polymyositis, dermatomyositis, and other forms of idiopathic inflammatory myopathy (Beyond the Basics)”); Rheumatoid arthritis (see “Patient education: Rheumatoid arthritis symptoms and diagnosis (Beyond the Basics)”); Oligoarticular juvenile chronic arthritis; Polyarteritis nodosum; Patients with organ-specific autoimmune diseases may also have a positive test for ANA. These diseases include: Thyroid diseases (Hashimoto's thyroiditis, Grave's disease); Gastrointestinal diseases (autoimmune hepatitis, primary biliary cholangitis [also known as primary biliary cirrhosis], inflammatory bowel disease); Pulmonary diseases (idiopathic pulmonary fibrosis). Patients with infectious diseases may also test positive for ANA. These diseases include: Viral infections (hepatitis C, parvovirus); Bacterial infections (tuberculosis); Parasitic infections (schistosomiasis).

Other associations with positive ANA tests have been noted, including: Various forms of cancer (relatively rare); as a harbinger of the future development of autoimmune disease; Various medications, without causing an autoimmune disease; Having one or more relatives with an autoimmune disease. Some individuals, even those without a relative with autoimmune disease, may have a positive test for ANA and yet never develop any autoimmune disease. ANAs are elevated in rosacea patients. A. Wozniacka et al. “Antinuclear antibodies in rosacea patients.” Postep Derm Alergol 2013; XXX, 1:1-5. DOI: 10.5114/pdia.2013.33372.

If a patient has a positive test for antinuclear antibodies (ANA), his or her health care provider, depending on the patient's symptoms or findings on physical examination, may order additional tests to identify specific types of autoantibodies. Some examples include:

Systemic lupus erythematosus. If a diagnosis of SLE is suspected, then additional tests, looking for autoantibodies directed against double-stranded DNA, Sm antigens, and ribosomal P antigens may be ordered. Because these antibodies are relatively specific for SLE, the results may provide important clues to facilitate the diagnosis of SLE. (See “Patient education: Systemic lupus erythematosus (SLE) (Beyond the Basics)”).

Sjögren's syndrome. If a diagnosis of Sjögren's syndrome is suspected, the health care provider may test for autoantibodies directed against antigens known as Ro/SSA and La/SSB. The presence of these autoantibodies provides support for the diagnosis of Sjögren's syndrome, a disorder which involves autoimmune destruction of the glands that produce tears and saliva.

Drug-induced systemic lupus erythematosus. If a diagnosis of drug-induced SLE is suspected, then a test for antihistone antibodies may be ordered. Antihistone antibodies are nearly always present in patients with drug-induced SLE. If antihistone antibodies are not detected, then the likelihood of this diagnosis (drug-induced SLE) is greatly reduced.

The methods provided herein are effective for a broad range of mites, depending on the application of interest. For example, the mite may be an unclassified mite. The mite may generally be a Demodex mite. The mite may be a specific mite species of the Demodex family. Demodex mites are ubiquitous and diverse, as suggested by recent molecular assessments of human samples, including phylogeny based on 18s rDNA sequences. Thoemmes et al. (2014) Ubiquity and Diversity of Human-Associated Demodex Mites. PLoS ONE 9(8): e106265. doi: 10.1371/journal.pone.0106265.

Exemplary mites that any of the methods provided herein are compatible with include any one or more of: Demodex aries, Demodex aurati, Demodex brevis, Demodex bovis, Demodex canis, Demodex caprae, Demodex caballi, Demodex cati, Demodex conicus, Demodex cornei, Demodex criceti, Demodex equi, Demodex folliculorum, Demodex foveolator, Demodex gapperi, Demodex gatoi, Demodex huttereri, Demodex injai, Demodex leucogasteri, Demodex microti, Demodex ovis, Demodex phyloides, Demodex ponderosus, Demodex vibrissae and Demodex zalophi.

Demodex mites, including Demodex folliculorum and Demodex brevis mites, may play a role in autoimmune conditions. An increased Demodex population has been observed in patients with inflammatory conditions. For most people, Demodex mites live harmlessly in the hair follicles, skin, eyes, eyelids, eyelashes, or meibomian lands as a result of either down-regulating host immunity or simply dodging host immune defenses. There is vociferous debate within the ophthalmology and dermatology community as to whether or not they are the causative agents of diseases such as acne vulgaris, rosacea, meibomian gland dysfunction, dry eye disease and blepharitis (inflammation of the eyelids).

Human beings are the one and only host of these two particular ubiquitous mites [1]. In fact, these two mites are considered to be the most common ectoparasite of humans [6]. Women tend to have a higher rate of Demodex infections [5]. The rate of infestation also seems to be correlated with age, with 84% of people at age 60 harboring mites and increasing to 100% in those 70 years and older [7]. Whether those that are immunocompromised are more susceptible to higher infestation rates is unknown, though some studies indicate that AIDs and leukemia patients may be more prone to greater than average numbers [5].

The mites are most commonly found in the scalp, face and upper chest area, with Demodex folliculorum exhibiting a predilection for the hair follicles and Demodex brevis for the sebaceous ducts and meibomian glands at the rim of the eyelids (the sebaceous ducts transfer the waxy sebum that lubricates the skin and hair from the sebum glands; the meibonmian glands are a special type of such gland) [4][5]. Demodex folliculorum are a communal bunch, tending to congregate in the follicle area of the hair or eyelashes with their posterior ends protruding from the follicular pores. Demodex brevis, on the other hand, tend to be more solitary and will occupy the sebaceous glands singly [6]. Both species are tiny, less than 0.4 mm, with elongated, clear bodies and four pairs of stout legs. Demodex brevis is usually a tad shorter, ˜0.1 mm, than Demodex folliculorum. They both have ridged scales along their cephalothorax and sharp, piercing teeth [6].

Short-lived creatures, a mite's life cycle from egg to larva to adult lasts from 14-18 days. Adults emerge from the follicles and ducts to reproduce at the surface of the skin where females will then deposit eggs in the sebaceous glands. Larva will mature via two nymphal stages in the glands until entering the follicles and ducts as adults to begin the cycle anew [6]. It is hypothesized that both species of mites feed upon sebum as a primary food source but may also munch on follicular and glandular epithelia. They are thought to be obligate ectoparasites, incapable of living outside their human host.

Some studies have discovered a greater than average mite density, greater than five mites per cm², do seem to play a role in skin diseases for patients [6]. Researchers have suggested that blockage of the hair follicles and sebaceous ducts by mites may result in epithelial hyperplasia, elicit a phagocytic, granulomatous reaction or bring about an inflammatory response due to their waste products [5]. The fact that treatment with certain antibiotics can reduce the severity of inflammatory conditions, rosacea, acne vulgaris, meibomian gland dysfunction, blepharitis and dry eye disease strongly suggests a microbial component to these mite-related diseases.

In 2007, researchers isolated from Demodex folliculorum a bacterium Bacillus oleronium that provoked inflammatory responses in 73% of rosacea patients but only 29% of controls [21]. These results suggest that patients with rosacea including ocular rosacea were sensitized to the bacteria and may be immunologically sensitive to the mites, bacteria or both [21].

Two antigenic proteins found on the bacterium's cell surface in particular appeared to be responsible for the inflammatory response by stimulating peripheral blood mononuclear cell proliferation one 83 kDa protein showed similarity with heat-shock proteins while the other 62 kDa protein shared amino acid sequence homology with a protease enzyme found to be involved signal transduction as well as carbohydrate metabolism [21]. Stronger proof of the pathogenic role of B. oleroniusm in ocular rosacea may also be found in the sensitivity of the bacterium to many antibiotics proven to be effective in the treatment of rosacea, specifically tetracycline, doxycycline and minocycline [21].

In an exemplary embodiment, an active agent is administered topically to a patient with an active ophthalmological afflictions condition in which the underlying cause is a Demodex mite. Because the target organisms, Demodex brevis and Demodex folliculorum, are ectoparasites in the mite family, an effective treatment must be capable of eradicating the entire lifecycle of such a microscopic insect, including egg, larval, and adult stages. For this reason, this embodiment treats such patients with several doses. Such spacing allows time for Demodex eggs to hatch into immature mites that are killed before they can mature into egg-producing adults. After the acetylcholinesterase inhibitor carries out its miticidal activity on Demodex brevis and Demodex folliculorum organisms, inflammatory responses to them begin to diminish but remnants of the dead mites still elicit some flushing and lesion formation until the cleanup processes of the body remove them, a process requiring six to twelve weeks. During this initial phase of acetylcholinesterase inhibitor administration, conventional autoimmune medications such as: anti-inflammatories, steroids and immunosuppressants

After prolonged intervals of freedom from symptoms, should classic signs begin to reappear, treatment can be repeated. The active agent can be formulated to ensure efficient transport to a sub-dermal layer. Because of the well-known barrier effect the skin presents to the penetration of topical medications, such a route of treatment with acetylcholinesterase inhibitor is anticipated to require once or twice daily applications for as long as twelve weeks to achieve sufficient follicle penetration and effective miticidal activity. A topical formulation that could achieve this effect would contain about 15% or less of the acetylcholinesterase inhibitor. The lesser the percentage of the acetylcholinesterase inhibitor that can be used while still receiving the miticidal effect and successfully treating the ocular condition is ideal for limiting any possible side effects of the chemical. Further, full facial body treatment is optionally useful for preventing reintroduction of the mites onto facial skin and glands.

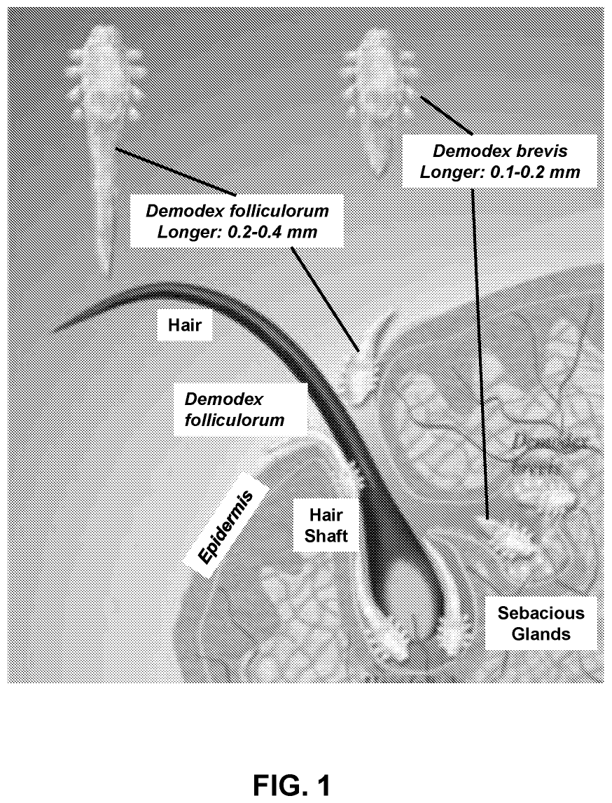

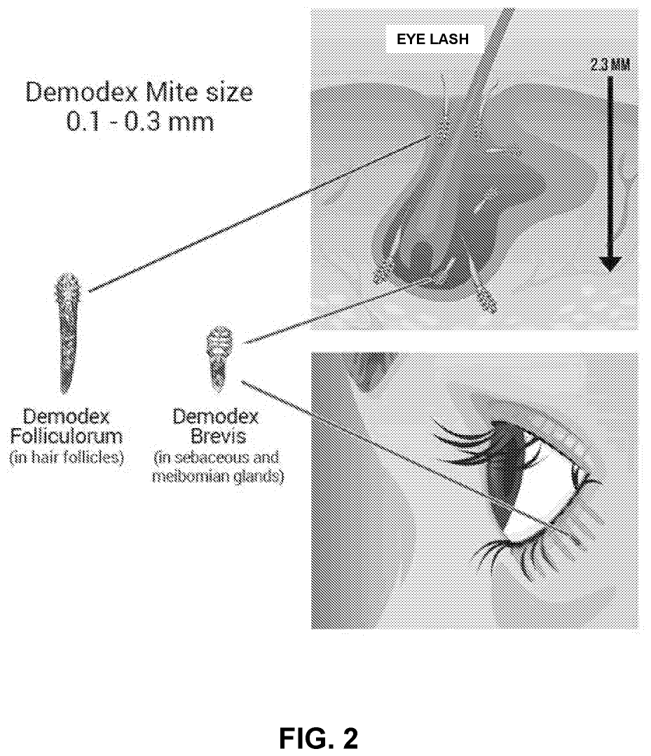

FIGS. 1-2 illustrate the demodex brevis and folliculorum mites in the skin area generally and the eye region, respectively, demonstrating the need to ensure treatment applications are able to achieve sub-dermal penetration.

### Medical Use of Carbamates:

Urethane (ethyl carbamate) was once produced commercially in the United States as an antineoplastic agent and for other medicinal purposes. It was found to be toxic and largely ineffective. It is occasionally used as a veterinary medicine.

In addition, some carbamates are used in human pharmacotherapy, for example, the cholinesterase inhibitors neostigmine and rivastigmine, whose chemical structure is based on the natural alkaloid physostigmine. Other examples are meprobamate and its derivatives like carisoprodol, felbamate, and tybamate, a class of anxiolytic and muscle relaxant drugs widely used in the 60s before the rise of benzodiazepines, and still used nowadays in some cases.

The cholinesterase inhibitors neostigmine and rivastigmine may be efficacious if they have similar miticidal capabilities compared to many other carbamate compounds.

Drug class and mechanism: Rivastigmine is an oral medication used to treat patients with Alzheimer's disease. Rivastigmine is in a class of drugs called cholinesterase inhibitors that also includes tacrine (Cognex), donepezil (Aricept), and galantamine (Razadyne—formerly known as Reminyl). Cholinesterase inhibitors inhibit (block) the action of acetylcholinesterase, the enzyme responsible for the destruction of acetylcholine. Acetylcholine is one of several neurotransmitters in the brain, chemicals that nerve cells use to communicate with one another. Reduced levels of acetylcholine in the brain are believed to be responsible for some of the symptoms of Alzheimer's disease.

Scientific Rationale:

Antimalarial drugs such as hydroxychloroquine (PLAQUENIL®) have been successfully used to treat lupus erythematosus, rheumatoid arthritis, juvenile idiopathic arthritis, systemic lupus, systemic sclerosis, juvenile and adult dermatomyositis, Sjögren's syndrome and porphyria cutanea tarda. These challenging diseases have no known etiology, but one interesting connection is that all can be treated with the anti-parasitic agent hydroxychloroquine (PLAQUENIL® antimalarial preparation). The antimalarial drug hydroxychloroquine has been hypothesized to be treating these autoimmune diseases as an immunosuppressing agent. Nobody has been able to clearly explain how hydroxychloroquine works as an immunosuppressant.

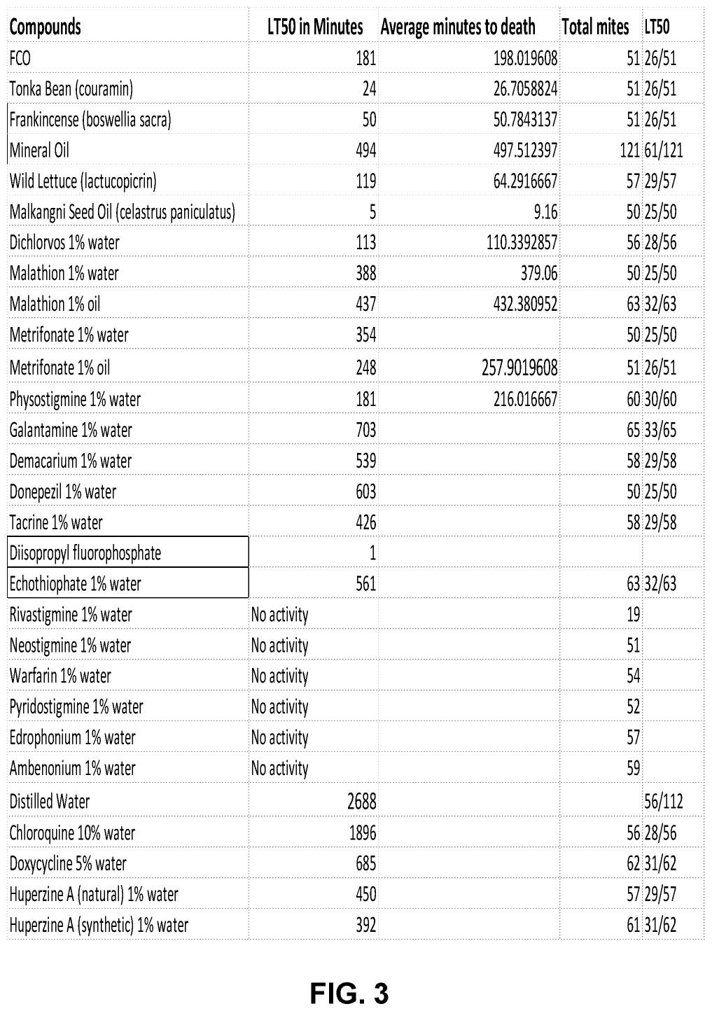

The anti-malarial drugs hydroxychloroquine and chloroquine treat autoimmune disease by acting as an anti-parasitic agent against Demodex. An in vitro assay establishes that the anti-malarial drug chloroquine has activity against Demodex mites, (FIG. 3). Accordingly, chloroquine and hydroxychloroquine may be used to treat autoimmune diseases, such as systemic lupus erythematosus, rheumatoid arthritis, juvenile idiopathic arthritis, systemic lupus, systemic sclerosis, juvenile and adult dermatomyositis, Sjögren's syndrome, and porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis by acting as an anti-parasitic against Demodex mites.

Example: A caucasian female suffering from lupus erythematosus that has previously responded to hydroxychloroquine therapy applied an acetylcholinesterase inhibitor to her malar rash. The patient self-reported the reduction in her lupus induced malar rash after several continued applications of the acetylcholinesterase inhibitor.

Proposed herein is the mechanism that hydroxychloroquine acts as an anti-parasitic agent against Demodex mites. The mite is ubiquitous on adult humans and appears to on-board the human epidermis sometime in or around puberty. This was discovered by searching for the mites' DNA signature on the human epidermis instead of the mite itself, which only presents itself at a rate of about 10% in skin biopsies. When looking at Demodex presence on the adult human epidermis, 99.9% of the time the DNA signature of the mite can be found by swabbing the skin and looking for the mites' DNA. Even more interesting is that only 70% of 18 year olds harbor mite DNA. This suggests that perhaps mite colonization of the human epidermis does not strictly occur vertically from parent to child. This could explain the mystery of why many autoimmune diseases onset in varying age groups.

Provided herein is treatment of these autoimmune diseases by treatment of the mites, including based on hydroxychloroquine evoking severe immune responses or allergic reactions to the Demodex mites, or via a pathogen harbored in or on the mites. We have previously repositioned the failed Alzheimer's disease drug, metrifonate, to treat papulopostular rosacea by targeting Demodex mites (a proven target for treating papulopostular rosacea). Metrifonate along with praziquantel has been used to treat the parasitic infection, schistosomiasis, for over 30 years. Both Praziquantil and hydroxychloroquine have been used to treat malaria by acting on the parasite, Plasmodium falciparum. Both metrifonate and hydroxychloroquine are postulated to possess anti-parasitic activity against Demodex mites and are treating cutaneous diseases by eliminating the mite or a pathogen on or inside the mites.

Antinuclear antibodies (ANAs) also known as antinuclear factor (AN), are autoantibodies that bind to contents of the cell nucleus. In normal healthy individuals, the immune system produces antibodies to foreign proteins (antigens) but not to human proteins (autoantigens). In some other individuals, antibodies to human antigens are produced.

There are many subtypes of ANAs and each of these antibody subtypes binds to different proteins or protein complexes within the nucleus. They are found in many disorders including autoimmunity and infection, with different prevalence of antibodies depending on the condition. This allows the use of ANAs in the diagnosis of some autoimmune disorders, including systemic lupus erythematosus, Sjögren's syndrome, scleroderma, mixed connective tissue disease, polymyositis, dermatomyositis, autoimmune hepatitis, and drug-induced lupus.

The ANA test detects the autoantibodies present in an individual's blood serum. The common tests used for detecting and quantifying ANAs are indirect immunofluorescence and enzyme-linked immunosorbent assay (ELISA). In immunofluorescence, the level of autoantibodies is reported as a titer. This is the highest dilution of the serum at which autoantibodies are still detectable. Positive autoantibody titers at a dilution equal to or greater than 1:160 are usually considered as clinically significant. ANAs are found in many disorders, as well as in some healthy individuals. These disorders include: systemic lupus erythematosus (SLE), rheumatoid arthritis, Sjögren's syndrome, scleroderma, polymyositis, dermatomyositis, primary biliary cirrhosis, drug induced lupus, autoimmune hepatitis, multiple sclerosis, discoid lupus, thyroid disease, antiphospholipid syndrome, juvenile idiopathic arthritis, psoriatic arthritis, juvenile dermatomyositis, idiopathic thrombocytopaenic purpura, and infection. These antibodies can be subdivided according to their specificity, and each subset has different propensities for specific disorders.

Coincidentally, out of all the autoimmune diseases (there are over 100), ANA diagnostics are used in confirming specifically the autoimmune diseases we previously identified as targets because they are being treated with the anti-parasitic, hydroxychloroquine. Statistically speaking, the odds of it being just coincidental that ANA diagnostics are being used in the diagnosis of autoimmune disease, where the anti-parasitic hydroxychloroquine is being effectively used as an intervention, are low.

Even more compelling is that in 2013, Woźniacka et al., reported in “Antinuclear Antibodies in Rosacea Patients”, that over a half (53.5%) of rosacea patients had an ANA titer greater than or equal to 1:160. Within this group 13.86% had a titer of 1:320, 8.91% had a titer of 1:640, and 6.93% had a titer of 1:1,280 or higher. The specificity of these antibodies could not be identified. Only two of 26 healthy volunteers had elevated ANA titers. One had a titer of 1:160 and the other of 1:320. The fact that elevated ANA titers are commonly found in rosacea patients where Demodex mites are being targeted as a causative agent, is a landmark discovery that's being ignored by the dermatology community.

In addition, the drugs that treat dry eye disease where Demodex is also now suspected in playing a causative role, are treated with the same agents that are being used to treat many of these autoimmune diseases. Cyclosporine and Lifitegrast work by downregulating the T cell or T lymphocytes of the patient. The immunosuppressant Tacrolimus has also been reported to treat dry eye disease. We believe these immunosuppressants are downregulating the host's immune response to the Demodex mites in many different autoimmune diseases; the diseases could be redefined as moderate to severe allergic reactions to Demodex mites or pathogens harbored by the mites.

Intense pulsed light (IPL) has also been used to treat rosacea and dry eye. Demodex has been implicated as playing a causative role in both diseases. IPL treatment has been proven to kill Demodex mites as reported by Prieto et al., in “Effects of intense pulsed light on sun-damaged human skin, routine, and ultrastructural analysis” in 2002. That work was also reported by Timothy Kirn in “Intense pulsed light eradicates Demodex mites”, published by Skin Allergy News in 2002. In 2000, Dr. J L Levy described treating chronic facial erythema of systemic lupus erythematosus in “Intense pulsed light treatment for chronic facial erythema of systemic lupus erythematosus: a case report.” In the report, a 33-year-old woman who had been diagnosed previously with systemic lupus erythematosus, presented with chronic erythema and rosacea of the face. The patient suffered from flushing and burning of the facial skin and sought prior treatment with antimalarial drugs. After various treatment options were discussed with the patient, she agreed to undergo intense pulsed light therapy. Improvement was noted after the first session and 75% clearance was observed at 1 month after a second session. There were no adverse effects associated with the treatment. One year later it was observed that the results of the two treatments had been maintained. Photosensitivity is one of the most widespread symptoms of lupus; UV rays either from the sun or from artificial light, are the most common reported lupus flare trigger. Using IPL in a lupus patient is very counterintuitive. The resulting clearance in the case report is now being hypothesized to be from the removal of the Demodex mite from the patient's facial skin. IPL is also currently being utilized to treat dry eye disease and again we believe this is by eliminating Demodex with IPL energy. Pulsed dye lasers are also used to treat rosacea, active acne vulgaris, and skin lesions caused by lupus erythematosus. We believe the pulse dye laser works beyond just vascular coagulation of telangiectasias in all three indications by eliminating the Demodex mite from the epidermis.

Many rosacea flare triggers overlap with autoimmune flare triggers especially those observed in lupus (SLE) patients. Lupus rash, much like the rash seen in rosacea patients, tends to occur in the T and U zones of the face. In lupus, the rash is referred to as a malar or butterfly rash. Antibiotics that treat rosacea are capable of inducing lupus, especially tetracyclines. It's our belief that the antibiotics are acting on the mite and causes the drug-induced lupus. The current most effective way of treating drug-induced lupus is to discontinue the medication that initiated the reaction. It should be noted that tetracycline antibiotics are used to treat acne, rosacea, and meibomian gland dysfunction, all disorders where Demodex are now being implicated. Whether the antibiotics act as anti-microbial agents and kill the Demodex or act on the mites' gut bacteria has yet to be fully elucidated.

Additionally, Demodex mites have been found in the lymphatic glands of dogs. Demodex canis could be used as a surrogate model to better understand Demodex brevis and Demodex folliculorum in humans. It should also be noted that dogs suffer from lupus, referred to as canine discoid lupus erythematosus, and it may be caused by Demodex canis. Human Demodex mites have also been found to harbor Microsporum canis in the human epidermis. Microsporum canis (M. canis) is a zoophilic dermatophyte, given that it typically colonizes the outer surface of an animal's body. Cats and dogs are believed to be the most common population hosts of this fungus, while humans are occasional hosts, in which the fungus can induce secondary infections. M. canis has been identified as a causal agent of a ringworm infection in pets called tinea capitis, and tinea corporis in humans (children in particular).

M. canis is among the most common dermatophytes associated with tinea capitis and tinea corporis. Unlike some dermatophyte species, M. canis typically does not cause large epidemics. Humans become infected as a result of direct or indirect contact with infected pets. M. canis generally invades hair and skin; however, some nail infections have been reported. When hair shafts are infected, M. canis causes an ectothrix-type infection where the fungus envelopes the exterior of the hair shaft without the formation of internal spores. This colonization of the hair shaft causes it to become unsheathed, resulting in characteristic round or oval non-inflammatory lesions that develop on the scalp. Infection triggers an acute leukocytic reaction in subcutaneous tissues, which gradually becomes highly inflammatory and leads to hair loss, in the case of tinea capitis. We believe Demodex is capable of harboring other, yet to be identified pathogens that play a causative role in many autoimmune diseases.

### Example 1: AChE Inhibitors

Lead clinical candidates include many acetylcholinesterase inhibitors (AChE Inhibitors) that can act on the AChE pathway inside the mite. AChE inhibiting compounds have known knockout effects on mites. This area has been heavily researched by the agricultural chemical industry. Based on these findings we can hypothesize AChE inhibitors will be effective potential agents in reducing Demodex mites in the human body. The potent AChE inhibitor, dichlorvos 1%, has already been used on one patient to achieve complete and continued remission of Papulopostular Rosacea (PPR) a disease that is believed to be caused by Demodex mites. PCT Pub. No. WO 2015/017328.

With respect to safety, AChE inhibitors have been studied extensively in medicine and are most commonly used as oral agents in the treatment of Alzheimer's Disease (AD). Many FDA-approved AChE inhibitors can be repositioned to treat autoimmune diseases by acting against Demodex. There are also failed AChE inhibitors, trialed to treat AD, as potential clinical candidates. The compounds went through Phase III studies to treat AD, but failed to meet efficacy endpoints for AD treatment. The safety profile from the studies with oral and intravenous AChE inhibitors in humans is extensive.

AChE Inhibitors applied topically to the eyelid appear to be clinical candidates with a remarkable safety profile. AChE inhibitors appear to have the potential to be a best-in-class treatment that leads to remission of these challenging autoimmune diseases in patients through continued prophylactic use. The low dosage of AChE inhibitors proposed to be administered, and the fact that the suggested delivery vehicle is a topical embodiment, versus the current oral and intravenous formulations, should be well received by the FDA. Ivermectin is the first treatment to clear the FDA that specifically targets Demodex mites to treat PPR (FDA clearance was received on Dec. 24, 2014), its safety is well documented.

### Example 2: Avermectins, Including Ivermectin

Ivermectin is derived from the avermectins, a class of highly active broad-spectrum, anti-parasitic agents isolated from the fermentation products of Streptomyces avermitilis. Ivermectin is a mixture containing at least 90% 5-O demethyl-22,23-dihydroavermectin Ala and less than 10% 5-O-demethyl-25-de(1-methylpropyl)-22,23-dihydro-25-(1-methylethyl)avermectin ₁ₐ, generally referred to as 22,23-dihydroavermectin B₁ₐ and B_{1b}, or H₂B₁ₐ and H₂B_{1b}, respectively. The respective empirical formulas are C₄₈H₇₄O₁₄ and C₄₇H₇₂O₁₄, with molecular weights of 875.10 and 861.07, respectively. The structural formulas are:

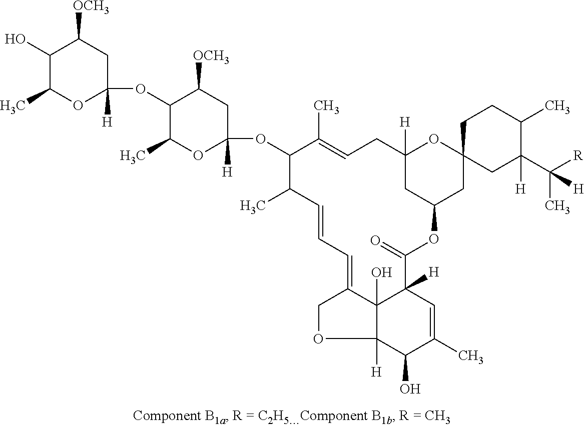

Any of the methods provided herein may use a compound that is from the avermectin family. Accordingly, in any of the claims provided herein, the specific compound ivermectin, may be replaced by the family of compounds known as avermectin, or any of the specific compounds of the avermectin family, including abamectin, doramectin, emamectin, ivermectin, selamectin.

### Example 3: Chloroquine and/or Hydroxychloroquine

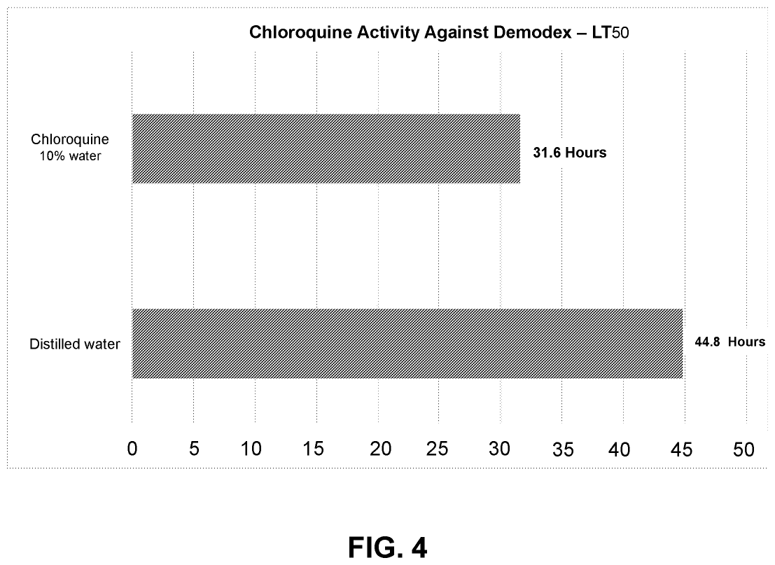

Any of the methods provided herein may use a compound that is chloroquine or hydroxychloroquine (see, e.g., FIG. 4).

Hydroxychloroquine was approved for medical use in the United States in the 1950s and is on the World Health Organization's List of Essential Medicines, with the chemical structure:

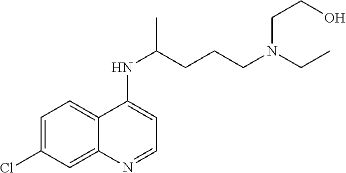 [SMILES: CCN(CCO)CCCC(C)Nc1ccnc2cc(Cl)ccc12]

Chloroquine has the structural formula:

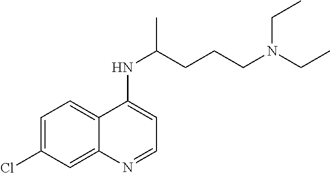 [SMILES: CCN(CC)CCCC(C)Nc1ccnc2cc(Cl)ccc12]

### Example: Administration and Formulation

Salts and Prodrugs: The invention contemplates pharmaceutically active compounds either chemically synthesized or formed by in vivo biotransformation to compounds set forth herein.

Compounds of this invention and compounds useful in the methods of this invention include those of the compounds and formula(s) described herein and pharmaceutically-acceptable salts and esters of those compounds. In embodiments, salts include any salts derived from the acids and bases of the formulas herein which are acceptable for use in human or veterinary applications. In embodiments, the term ester refers to hydrolyzable esters of compounds of the names and formulas herein. In embodiments, salts and esters of the compounds of the formulas herein can include those which have the same or better therapeutic, diagnostic, or pharmaceutical (human or veterinary) general properties as the compounds of the formulas herein. In an embodiment, a composition of the invention is a compound or salt or ester thereof suitable for pharmaceutical formulations.

Compounds of the invention and used in the methods of the invention can have prodrug forms. Prodrugs of the compounds of the invention are useful in embodiments including compositions and methods. Any compound that will be converted in vivo to provide a biologically, pharmaceutically, diagnostically, or therapeutically active form of a compound of the invention is a prodrug. Various examples and forms of prodrugs are well known in the art. Examples of prodrugs are found, inter alia, in: Design of Prodrugs, edited by H. Bundgaard, (Elsevier, 1985); Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191 (1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392). A prodrug, such as a pharmaceutically acceptable prodrug, can represent prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of a compound described herein, for example, by hydrolysis in blood or by other cell, tissue, organ, or system processes. Further discussion is provided in: T. Higuchi and V. Stella, Pro-drugs as Novel Delivery Systems, V. 14 of the A.C.S. Symposium Series; and in Edward B. Roche, ed., Bioreversible Carriers in Drug Design, American Pharmaceutical Association and Pergamon Press (1987).

The active agent may also comprise quinine, such as having the following chemical structure, and salts thereof:

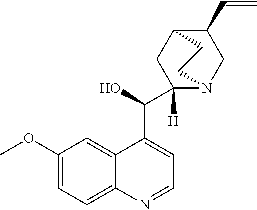 [SMILES: C=C[C@H]1C[N@]2CC[C@H]1C[C@H]2[C@H](O)c1ccnc2ccc(OC)cc12]

Quinine may be provided as a salt. It may also be provided in various related formulations, including the hydrochloride, dihydrochloride, sulfate, bisulfate and gluconate. Quinine salts may be given orally or intravenously (IV); quinine gluconate may also be given intramuscularly (IM) or rectally (PR).

Active ingredients of the invention can be formulated with pharmaceutically-acceptable anions and/or cations. Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include, among others, halides (e.g., F⁻, Cl⁻, Br⁻, At⁻), sulfate, acetates (e.g., acetate, trifluoroacetate), ascorbates, aspartates, benzoates, citrates, and lactate.

Pharmaceutically acceptable salts comprise pharmaceutically-acceptable anions and/or cations. As used herein, the term “pharmaceutically acceptable salt” can refer to acid addition salts or base addition salts of the compounds in the present disclosure. A pharmaceutically acceptable salt is any salt which retains at least a portion of the activity of the parent compound and does not impart significant deleterious or undesirable effect on a subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts can be derived from amino acids, including, but not limited to, cysteine. Other pharmaceutically acceptable salts can be found, for example, in Stahl et al., Handbook of Pharmaceutical Salts: Properties, Selection, and Use, Wiley-VCH, Verlag Helvetica Chimica Acta, Zurich, 2002. (ISBN 3-906390-26-8).

Efficacy: Typically, a compound of the invention, or pharmaceutically acceptable salt thereof, is administered to a subject in a diagnostically or therapeutically effective amount. One skilled in the art generally can determine an appropriate dosage.

Compositions for oral administration can be, for example, prepared in a manner such that a single dose in one or more oral preparations contains at least about 20 mg of the present compound per square meter of subject body surface area, or at least about 50, 100, 150, 200, 300, 400, or 500 mg of the present compound per square meter of subject body surface area (the average body surface area for a human is, for example, 1.8 square meters). In particular, a single dose of a composition for oral administration can contain from about 20 to about 600 mg, and in certain aspects from about 20 to about 400 mg, in another aspect from about 20 to about 300 mg, and in yet another aspect from about 20 to about 200 mg of the present compound per square meter of subject body surface area. Compositions for parenteral administration can be prepared in a manner such that a single dose contains at least about 20 mg of the present compound per square meter of subject body surface area, or at least about 40, 50, 100, 150, 200, 300, 400, or 500 mg of the present compound per square meter of subject body surface area. In particular, a single dose in one or more parenteral preparations contains from about 20 to about 500 mg, and in certain aspects from about 20 to about 400 mg, and in another aspect from about 20 to about 450 mg, and in yet another aspect from about 20 to about 350 mg of the present compound per square meter of subject body surface area. It should be recognized that these oral and parenteral dosage ranges represent generally preferred dosage ranges, and are not intended to limit the invention. The dosage regimen actually employed can vary widely, and, therefore, can deviate from the generally preferred dosage regimen. It is contemplated that one skilled in the art will tailor these ranges to the individual subject.

Toxicity and therapeutic efficacy of such compounds and bioconjugates can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compounds and bioconjugates that exhibit large therapeutic indices are preferred. While compounds and bioconjugates exhibiting toxic side effects can be used, care should be taken to design a delivery system that targets such compounds and bioconjugates to the site affected by the disease or disorder in order to minimize potential damage to unaffected cells and reduce side effects.

Data obtained from the cell culture assays and animal studies can be used in formulating a range of dosages for use in humans and other mammals. The dosage of such compounds and bioconjugates lies preferably within a range of circulating plasma or other bodily fluid concentrations that include the ED₅₀ and provides clinically efficacious results (i.e., reduction in disease symptoms). The dosage can vary within this range depending upon the dosage form employed and the route of administration utilized. For any compound and bioconjugate of the present invention, the therapeutically effective amount can be estimated initially from cell culture assays. A dosage can be formulated in animal models to achieve a circulating plasma concentration range that includes the ED₅₀ (the concentration of the test compound that achieves a half-maximal inhibition of symptoms) as determined in cell culture. Such information can be used to more accurately determine useful dosages in humans and other mammals. Compound and bioconjugate levels in plasma can be measured, for example, by high performance liquid chromatography.

An amount of a compound or bioconjugate that can be combined with a pharmaceutically acceptable carrier to produce a single dosage form will vary depending upon the patient treated and the particular mode of administration. It will be appreciated by those skilled in the art that the unit content of a compound/bioconjugate contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the particular purpose to be achieved according to the determination of those skilled in the art.

The dosage and dosage regime for treating a disease or condition can be selected in accordance with a variety of factors, including the type, age, weight, sex, diet and/or medical condition of the patient, the route of administration, pharmacological considerations such as activity, efficacy, pharmacokinetic and/or toxicology profiles of the particular compound/bioconjugate employed, whether a compound/bioconjugate delivery system is utilized, and/or whether the compound/bioconjugate is administered as a pro-drug or part of a drug combination. Thus, the dosage regime actually employed can vary widely from subject to subject, or disease to disease and different routes of administration can be employed in different clinical settings.

The identified compounds/bioconjugates monitor, treat, inhibit, control and/or prevent, or at least partially arrest or partially prevent, diseases and conditions of interest and can be administered to a subject at therapeutically effective amounts and optionally diagnostically effective amounts. Compositions/formulations of the present invention comprise a therapeutically effective amount (which can optionally include a diagnostically effective amount) of at least one compound or bioconjugate of the present invention. Subjects receiving treatment that includes a compound/bioconjugate of the invention are preferably animals (e.g., mammals, reptiles and/or avians), more preferably humans, horses, cows, dogs, cats, sheep, pigs, and/or chickens, and most preferably humans.

Administration: The preferred composition depends on the route of administration. Any route of administration can be used as long as the target of the compound or pharmaceutically acceptable salt is available via that route. Suitable routes of administration include, for example, oral, intravenous, parenteral, inhalation, rectal, nasal, topical (e.g., transdermal and intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual, and intestinal administration.

In an embodiment, the invention provides a method for treating a medical condition comprising administering to a subject (e.g. patient) in need thereof, a therapeutically effective amount of a composition of the invention, such as an avermectin or ivermectin composition. In an embodiment, the invention provides a method for diagnosing or aiding in the diagnosis of a medical condition comprising administering to a subject in need thereof, a diagnostically effective amount of a composition of the invention. In an embodiment, the medical condition is an autoimmune disease.

The diagnostic and therapeutic formulations of this invention can be administered alone, but can be administered with a pharmaceutical carrier selected upon the basis of the chosen route of administration and standard pharmaceutical practice.

Any suitable form of administration can be employed in connection with the diagnostic and therapeutic formulations of the invention. The diagnostic and therapeutic formulations of this invention can be administered intravenously, in oral dosage forms, intraperitoneally, subcutaneously, or intramuscularly, all using dosage forms well known to those of ordinary skill in the pharmaceutical arts.

The present compositions, preparations and formulations can be formulated into diagnostic or therapeutic compositions for enteral, parenteral, topical, aerosol, inhalation, or cutaneous administration. Topical or cutaneous delivery of the compositions, preparations and formulations can also include aerosol formulation, creams, gels, solutions, etc. The present compositions, preparations and formulations are administered in doses effective to achieve the desired diagnostic and/or therapeutic effect. Such doses can vary widely depending upon the particular compositions employed in the composition, the organs or tissues to be examined, the equipment employed in the clinical procedure, the efficacy of the treatment achieved, and the like. These compositions, preparations and formulations contain an effective amount of the composition(s), along with conventional pharmaceutical carriers and excipients appropriate for the type of administration contemplated. These compositions, preparations and formulations can also optionally include stabilizing agents and skin penetration enhancing agents.

(i) Parenteral Administration: Compounds and bioconjugates of the present invention can be formulated for parenteral administration by injection (e.g., by bolus injection or continuous infusion). Formulations for injection can be presented in unit dosage form in ampoules or in multi-dose containers with an optional preservative added. The parenteral preparation can be enclosed in ampoules, disposable syringes or multiple dose vials made of glass, plastic or the like. The formulation can take such forms as suspensions, solutions or emulsions in oily or aqueous vehicles, and can contain formulatory agents such as suspending, stabilizing and/or dispersing agents.

For example, a parenteral preparation can be a sterile injectable solution or suspension in a nontoxic parenterally acceptable diluent or solvent (e.g., as a solution in 1,3-butanediol). Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid can be used in the parenteral preparation.

Alternatively, compounds and bioconjugates of the present invention can be formulated in powder form for constitution with a suitable vehicle, such as sterile pyrogen-free water, before use. For example, a compound/bioconjugate suitable for parenteral administration can include a sterile isotonic saline solution containing between 0.1 percent and 90 percent weight per volume of the compound/bioconjugate. By way of example, a solution can contain from about 5 percent to about 20 percent, more preferably from about 5 percent to about 17 percent, more preferably from about 8 to about 14 percent, and still more preferably about 10 percent weight per volume of the compound/bioconjugate. The solution or powder preparation can also include a solubilizing agent and a local anesthetic such as lignocaine to ease pain at the site of the injection. Other methods of parenteral delivery of compounds/bioconjugates will be known to the skilled artisan and are within the scope of the invention.

(ii) Oral Administration: For oral administration, a compound/bioconjugate of the invention can be formulated to take the form of tablets or capsules prepared by conventional means with one or more pharmaceutically acceptable carriers (e.g., excipients such as binding agents, fillers, lubricants and disintegrants).

(iii) Controlled-Release Administration: Controlled-release (or sustained-release) preparations can be formulated to extend the activity of a compound/bioconjugate and reduce dosage frequency. Controlled-release preparations can also be used to effect the time of onset of action or other characteristics, such as blood levels of the compound/bioconjugate, and consequently affect the occurrence of side effects.

Controlled-release preparations can be designed to initially release an amount of a compound/bioconjugate that produces the desired therapeutic effect, and gradually and continually release other amounts of the compound/bioconjugate to maintain the level of therapeutic effect over an extended period of time. In order to maintain a near-constant level of a compound/bioconjugate in the body, the compound/bioconjugate can be released from the dosage form at a rate that will replace the amount of compound/bioconjugate being metabolized and/or excreted from the body. The controlled-release of a compound/bioconjugate can be stimulated by various inducers, e.g., change in pH, change in temperature, enzymes, water, and/or other physiological conditions or molecules.

Controlled-release systems can include, for example, an infusion pump which can be used to administer the compound/bioconjugate in a manner similar to that used for delivering insulin or chemotherapy to the body generally, or to specific organs or tumors. Typically, using such a system, the compound/bioconjugate is administered in combination with a biodegradable, biocompatible polymeric implant that releases the compound/bioconjugate over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target (e.g., organ, tissue, or group of cells), thus requiring only a fraction of a systemic dosage.

Compounds/bioconjugates of the invention can be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of compounds/bioconjugates will be known to the skilled artisan and are within the scope of the invention.

(iv) Inhalation Administration: Compounds/bioconjugates of the invention can be administered directly to the lung of a patient/subject by inhalation. For administration by inhalation, a compound/bioconjugate can be conveniently delivered to the lung by a number of different devices. For example, a Metered Dose Inhaler (“MDI”) which utilizes canisters that contain a suitable low boiling point propellant, e.g., dichlorodifluoromethane, trichlorofluoromethane, dichlorotetrafluoroethane, carbon dioxide or other suitable gas can be used to deliver a compound/bioconjugate directly to the lung. MDI devices are available from a number of suppliers such as 3M Corporation, Aventis, Boehringer Ingleheim, Forest Laboratories, GlaxoSmithKline, Merck & Co. and Vectura.

Alternatively, a Dry Powder Inhaler (DPI) device can be used to administer a compound/bioconjugate to the lung. DPI devices typically use a mechanism such as a burst of gas to create a cloud of dry powder inside a container, which can then be inhaled by the patient. DPI devices are also well known in the art and can be purchased from a number of vendors which include, for example, GlaxoSmithKline, Nektar Therapeutics, Innovata and Vectura. A popular variation is the multiple dose DPI (“MDDPI”) system, which allows for the delivery of more than one therapeutic dose. MDDPI devices are available from companies such as AstraZeneca, GlaxoSmithKline, TEVA, Merck & Co., SkyePharma and Vectura. For example, capsules and cartridges of gelatin for use in an inhaler or insufflator can be formulated containing a powder mix of the compound/bioconjugate and a suitable powder base such as lactose or starch for these systems.

Another type of device that can be used to deliver a compound/bioconjugate to the lung is a liquid spray device supplied, for example, by Aradigm Corporation. Liquid spray systems use extremely small nozzle holes to aerosolize liquid compound/bioconjugate formulations that can then be directly inhaled into the lung. For example, a nebulizer device can be used to deliver a compound/bioconjugate to the lung. Nebulizers create aerosols from liquid compound/bioconjugate formulations by using, for example, ultrasonic energy to form fine particles that can be readily inhaled. Examples of nebulizers include devices supplied by Aventis and Battelle.

In another example, an electrohydrodynamic (“EHD”) aerosol device can be used to deliver a compound/bioconjugate to the lung. EHD aerosol devices use electrical energy to aerosolize liquid compound/bioconjugate solutions or suspensions. The electrochemical properties of the compound/bioconjugate formulation are important parameters to optimize when delivering this compound/bioconjugate to the lung with an EHD aerosol device. Such optimization is routinely performed by one of skill in the art. Other methods of intra-pulmonary delivery of compounds/bioconjugates will be known to the skilled artisan and are within the scope of the invention.

Liquid compound/bioconjugate formulations suitable for use with nebulizers and liquid spray devices and EHD aerosol devices will typically include the compound/bioconjugate with a pharmaceutically acceptable carrier. In one exemplary embodiment, the pharmaceutically acceptable carrier is a liquid such as alcohol, water, polyethylene glycol or a perfluorocarbon. Optionally, another material can be added to alter the aerosol properties of the solution or suspension of the compound/bioconjugate. For example, this material can be a liquid such as an alcohol, glycol, polyglycol or a fatty acid. Other methods of formulating liquid compound/bioconjugate solutions or suspensions suitable for use in aerosol devices are known to those of skill in the art.

(v) Depot Administration: A compound/bioconjugate of the invention can be formulated as a depot preparation. Such long-acting formulations can be administered by implantation (e.g., subcutaneously or intramuscularly) or by intramuscular injection. Accordingly, the compound/bioconjugate can be formulated with suitable polymeric or hydrophobic materials such as an emulsion in an acceptable oil or ion exchange resin, or as sparingly soluble derivatives such as a sparingly soluble salt. Other methods of depot delivery of compounds/bioconjugates will be known to the skilled artisan and are within the scope of the invention.

(vi) Topical Administration: For topical application, a compound/bioconjugate can be combined with a pharmaceutically acceptable carrier so that an effective dosage is delivered, based on the desired activity ranging from an effective dosage, for example, of 1.0 μM to 1.0 mM. In one aspect of the invention, a topical formulation of a compound/bioconjugate can be applied to the skin. The pharmaceutically acceptable carrier can be in the form of, for example, and not by way of limitation, an ointment, cream, gel, paste, foam, aerosol, suppository, pad or gelled stick.

A topical formulation can include a therapeutically effective amount of a compound/bioconjugate in an ophthalmologically acceptable excipient such as buffered saline, mineral oil, vegetable oils such as corn or arachis oil, petroleum jelly, Miglyol 182, alcohol solutions, or liposomes or liposome-like products. Any of these formulations of such compounds/bioconjugates can include preservatives, antioxidants, antibiotics, immunosuppressants, and other biologically or pharmaceutically effective agents that do not exert a significant detrimental effect on the compound/bioconjugate. Other methods of topical delivery of compounds/bioconjugates will be known to the skilled artisan and are within the scope of the invention. Topical formulations of the invention further include those comprising one or more compositions useful for penetrating the skin, such as dimethyl sulfoxide (DMSO).

(vii) Rectal Administration: Compounds/bioconjugates of the invention can be formulated in rectal formulations such as suppositories or retention enemas that include conventional suppository bases such as cocoa butter or other glycerides and/or binders and/or carriers such as triglycerides, microcrystalline cellulose, gum tragacanth or gelatin. Rectal formulations can contain a compound/bioconjugate in the range of 0.5% to 10% by weight, for example. Other methods of rectal delivery of compounds/bioconjugates will be known to the skilled artisan and are within the scope of the invention.

(viii) Other Systems of Administration: Various other delivery systems are known in the art and can be used to administer the compounds/bioconjugates of the invention. Moreover, these and other delivery systems can be combined and/or modified to promote optimization of the administration of compounds/bioconjugates of the present invention. Exemplary formulations that include compounds/bioconjugates of the present invention are described elsewhere herein (the compounds/bioconjugates of the present invention are indicated as the active ingredient, but those of skill in the art will recognize that pro-drugs and compound combinations are also meant to be encompassed by this term).

5.d: Formulation: In an embodiment, the invention provides a medicament which comprises a therapeutically effective amount of one or more compositions of the invention, such as chloroquine and/or hydroxychloroquine. In an embodiment, the invention provides a medicament which comprises a diagnostically effective amount of one or more compositions of the invention. In an embodiment, the invention provides a method for making a medicament for treatment of a condition described herein, such as the treatment of a skin condition or dermatological disease. In an embodiment, the invention provides a method for making a medicament for diagnosis or aiding in the diagnosis of a condition described herein, such as the diagnosis of a skin condition or dermatological disease. In an embodiment, the invention provides the use of one or more compositions set forth herein for the making of a medicament for the treatment of a skin condition or dermatological disease. In an embodiment, the invention provides the use of one or more compositions set forth herein for the treatment of a disease. In an embodiment, the invention provides the use of one or more compositions set forth herein for the diagnosis of a disease. Compositions of the invention include formulations and preparations comprising one or more of chloroquine and/or hydroxychloroquine provided in an aqueous solution, such as a pharmaceutically acceptable formulation or preparation. Optionally, compositions of the invention further comprise one or more pharmaceutically acceptable surfactants, buffers, electrolytes, salts, carriers, binders, coatings, preservatives and/or excipients.

In an embodiment, the invention provides a pharmaceutical formulation having an active ingredient comprising a composition of the invention, such as chloroquine and/or hydroxychloroquine. In an embodiment, the invention provides a method of synthesizing a composition of the invention or a pharmaceutical formulation thereof, such as an chloroquine and/or hydroxychloroquine. In an embodiment, a pharmaceutical formulation comprises one or more excipients, carriers, diluents, and/or other components as would be understood in the art. Preferably, the components meet the standards of the National Formulary (“NF”), United States Pharmacopoeia (“USP”; United States Pharmacopeial Convention Inc., Rockville, Md.), or Handbook of Pharmaceutical Manufacturing Formulations (Sarfaraz K. Niazi, all volumes, ISBN: 9780849317521, ISBN 10: 0849317525; CRC Press, 2004). See, e.g., United States Pharmacopeia and National Formulary (USP 30-NF 25), Rockville, Md.: United States Pharmacopeial Convention (2007 and 2008), and each of any earlier editions; The Handbook of Pharmaceutical Excipients, published jointly by the American Pharmacists Association and the Pharmaceutical Press (Pharmaceutical Press (2005) (ISBN-10: 0853696187, ISBN-13: 978-0853696186)); Merck Index, Merck & Co., Rahway, N.J.; and Gilman et al., (eds) (1996); Goodman and Gilman's: The Pharmacological Bases of Therapeutics, 8th Ed., Pergamon Press. In embodiments, the formulation base of the formulations of the invention comprises physiologically acceptable excipients, namely, at least one binder and optionally other physiologically acceptable excipients. Physiologically acceptable excipients are those known to be usable in the pharmaceutical technology sectors and adjacent areas, particularly, those listed in relevant pharmacopeias (e.g. DAB, Ph. Eur., BP, NF, USP), as well as other excipients whose properties do not impair a physiological use.

This invention also is directed, in part, to pharmaceutical compositions including a therapeutically effective amount of a compound or salt of this invention, as well as processes for making such compositions. Such compositions generally include one or more pharmaceutically acceptable carriers (e.g., excipients, vehicles, auxiliaries, adjuvants, diluents) and can include other active ingredients. Formulation of these compositions can be achieved by various methods known in the art. A general discussion of these methods can be found in, for example, Hoover, John E., Remington's Pharmaceutical Sciences (Mack Publishing Co., Easton, Pa.: 1975). See also, Lachman, L., eds., Pharmaceutical Dosage Forms (Marcel Decker, New York, N.Y., 1980).

The diagnostic and therapeutic formulations of this invention and medicaments of this invention can further comprise one or more pharmaceutically acceptable carriers, excipients, buffers, emulsifiers, surfactants, electrolytes or diluents. Such compositions and medicaments are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985).

Compositions of the invention include formulations and preparations comprising one or more of the present compounds provided in an aqueous solution, such as a pharmaceutically acceptable formulation or preparation. Optionally, compositions of the invention further comprise one or more pharmaceutically acceptable surfactants, buffers, electrolytes, salts, carriers, binders, coatings, preservatives and/or excipients.

Compounds and bioconjugates of the present invention can be formulated by known methods for administration to a subject using several routes which include, but are not limited to, parenteral, oral, topical, intradermal, intramuscular, intraperitoneal, intravenous, subcutaneous, intranasal, epidural, and ophthalmic routes. An individual compound/bioconjugate can be administered in combination with one or more additional compounds/bioconjugates of the present invention and/or together with other biologically active or biologically inert agents. Such biologically active or inert agents can be in fluid or mechanical communication with the compound(s)/bioconjugate(s) or attached to the compound(s)/bioconjugate(s) by ionic, covalent, Van der Waals, hydrophobic, hydrophilic or other physical forces. It is preferred that administration is localized in a subject, but administration can also be systemic.

Compounds and bioconjugates of the present invention can be formulated by any conventional manner using one or more pharmaceutically acceptable carriers. Thus, the compound(s)/bioconjugate(s) and their pharmaceutically acceptable salts and solvates can be specifically formulated for administration, e.g., by inhalation or insufflation (either through the mouth or the nose) or oral, buccal, parenteral or rectal administration. The compounds/bioconjugates can take the form of charged, neutral and/or other pharmaceutically acceptable salt forms. Examples of pharmaceutically acceptable carriers include, but are not limited to, those described in REMINGTON'S PHARMACEUTICAL SCIENCES (A.R. Gennaro, Ed.), 20th edition, Williams & Wilkins PA, USA (2000).

Compounds and bioconjugates of the present invention can be formulated in the form of solutions, suspensions, emulsions, tablets, pills, capsules, powders, controlled- or sustained-release formulations and the like. Such formulations will contain a therapeutically effective amount of the compound/bioconjugate, preferably in purified form, together with a suitable amount of carrier so as to provide the form for proper administration to the patient. The formulation should suit the mode of administration.

Pharmaceutically acceptable carriers that can be used in conjunction with the compounds of the invention are well known to those of ordinary skill in the art. Carriers can be selected based on a number of factors including, for example, the particular compound(s) or pharmaceutically acceptable salt(s) used; the compound's concentration, stability, and intended bioavailability; the condition being treated; the subject's age, size, and general condition; the route of administration; etc. A general discussion related to carriers can be found in, for example, J. G. Nairn, Remington's Pharmaceutical Science, pp. 1492-1517 (A. Gennaro, ed., Mack Publishing Co., Easton, Pa. (1985)).

Solid dosage forms for oral administration include, for example, capsules, tablets, gel-caps, pills, dragees, troches, powders, granules, and lozenges. In such solid dosage forms, the compounds or pharmaceutically acceptable salts thereof can be combined with one or more pharmaceutically acceptable carriers. The compounds and pharmaceutically acceptable salts thereof can be mixed with carriers including, but not limited to, lactose, sucrose, starch powder, corn starch, potato starch, magnesium carbonate, microcrystalline cellulose, cellulose esters of alkanoic acids, cellulose alkyl esters, talc, stearic acid, magnesium stearate, magnesium oxide, sodium and calcium salts of phosphoric and sulfuric acids, sodium carbonate, agar, mannitol, sorbitol, sodium saccharin, gelatin, acacia gum, alginic acid, sodium alginate, tragacanth, colloidal silicon dioxide, croscarmellose sodium, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can include buffering agents, such as sodium citrate, or magnesium or calcium carbonate or bicarbonate. Tablets and pills additionally can, for example, include a coating (e.g., an enteric coating) to delay disintegration and absorption. The concentration of the present compounds in a solid oral dosage form can be from about 5 to about 50% for example, and in certain aspects from about 8 to about 40%, and in another aspect from about 10 to about 30% by weight based on the total weight of the composition.

Liquid dosage forms of the compounds of the invention for oral administration include, for example, pharmaceutically acceptable emulsions, solutions, suspensions, syrups, and elixirs containing inert diluents commonly used in the art (e.g., water). Such compositions also can include adjuvants, such as wetting, emulsifying, suspending, flavoring (e.g., sweetening), and/or perfuming agents. The concentration of the present compounds in the liquid dosage form can be from about 0.01 to about 5 mg, and in certain aspects from about 0.01 to about 1 mg, and in another aspect from about 0.01 to about 0.5 mg per ml of the composition. Low concentrations of the compounds of the invention in liquid dosage form can be prepared in the case that the compound is more soluble at low concentrations. Techniques for making oral dosage forms useful in the invention are generally described in, for example, Modern Pharmaceutics, Chapters 9 and 10 (Banker & Rhodes, Editors (1979)). See also, Lieberman et al., Pharmaceutical Dosage Forms: Tablets (1981). See also, Ansel, Introduction to Pharmaceutical Dosage Forms (2nd Edition (1976)).

In some aspects of the invention, tablets or powders for oral administration can be prepared by dissolving the compound in a pharmaceutically acceptable solvent capable of dissolving the compound to form a solution and then evaporating when the solution is dried under vacuum. A carrier can also be added to the solution before drying. The resulting solution can be dried under vacuum to form a glass. The glass can then be mixed with a binder to form a powder. This powder can be mixed with fillers or other conventional tableting agents, and then processed to form a tablet. Alternatively, the powder can be added to a liquid carrier to form a solution, emulsion, suspension, or the like.

In some aspects, solutions for oral administration are prepared by dissolving the compound in a pharmaceutically acceptable solvent capable of dissolving the compound to form a solution. An appropriate volume of a carrier is added to the solution while stirring to form a pharmaceutically acceptable solution for oral administration.

In some embodiments, a liposome or micelle can be utilized as a carrier or vehicle for the composition. For example, in some embodiments, the compound can be a part of the lipophilic bilayers or micelle, and the targeting ligand, if present, can be on the external surface of the liposome or micelle. As another example, a targeting ligand can be externally attached to the liposome or micelle after formulation for targeting the liposome or micelle (which contains the chloroquine and/or hydroxychloroquine agents) to the desired tissue, organ, or other site in the body.

Injectable preparations (e.g., sterile injectable aqueous or oleaginous suspensions) can be formulated according to the known art using suitable dispersing, wetting agents, and/or suspending agents. Acceptable vehicles for parenteral use include both aqueous and nonaqueous pharmaceutically-acceptable solvents. Suitable pharmaceutically acceptable aqueous solvents include, for example, water, saline solutions, dextrose solutions (such as DW5), electrolyte solutions, etc.

In one embodiment, the present compounds are formulated as nanoparticles or microparticles. Use of such nanoparticle or microparticle formulations can be beneficial for some applications to enhance delivery, localization, target specificity, administration, etc. of the compound. Potentially useful nanoparticles and microparticles include, but are not limited to, micelles, liposomes, microemulsions, nanoemulsions, vesicles, tubular micelles, cylindrical micelles, bilayers, folded sheets structures, globular aggregates, swollen micelles, inclusion complex, encapsulated droplets, microcapsules, nanocapsules or the like. As will be understood by those having skill in the art, the present compounds can be located inside the nanoparticle or microparticle, within a membrane or wall of the nanoparticle or microparticle, or outside of (but bonded to or otherwise associated with) the nanoparticle or microparticle. The agent formulated in nanoparticles or microparticles can be administered by any of the routes previously described. In a formulation applied topically, the compound is slowly released over time. In an injectable formulation, the liposome, micelle, capsule, etc., circulates in the bloodstream and is delivered to the desired site (e.g., target tissue).

Preparation and loading of nanoparticles and microparticles are well known in the art. As one example, liposomes can be prepared from dipalmitoyl phosphatidylcholine (DPPC) or egg phosphatidylcholine (PC) because this lipid has a low heat transition. Liposomes are made using standard procedures as known to one skilled in the art (e.g., Braun-Falco et al., (Eds.), Griesbach Conference, Liposome Dermatics, Springer-Verlag, Berlin (1992), pp. 69 81; 91 117. Polycaprolactone, poly(glycolic) acid, poly(lactic) acid, polyanhydride or lipids can be formulated as microspheres. As an illustrative example, the present compounds can be mixed with polyvinyl alcohol (PVA), the mixture then dried and coated with ethylene vinyl acetate, then cooled again with PVA. In a liposome, the present compounds can be within one or both lipid bilayers, in the aqueous between the bilayers, or within the center or core. Liposomes can be modified with other molecules and lipids to form a cationic liposome. Liposomes can also be modified with lipids to render their surface more hydrophilic which increases their circulation time in the bloodstream. The thus-modified liposome has been termed a “stealth” liposome, or a long-lived liposome, as described in U.S. Pat. No. 6,258,378, and in Stealth Liposomes, Lasic and Martin (Eds.) 1995 CRC Press, London. Encapsulation methods include detergent dialysis, freeze drying, film forming, injection, as known to one skilled in the art and disclosed in, for example, U.S. Pat. No. 6,406,713. Optionally, the present compositions and methods include a micelle delivery system, for example, involving one or more PEG-based amphiphilic polymers developed for drug delivery including: PEG-poly(ε-caprolactone), PEG-poly(amino acid), PEG-polylactide or PEG-phospholipid constructs; a cross linked poly(acrylic acid) polymer system, a phospholipid-based system and/or block copolymer systems comprising one or more of the following polymer blocks: a poly(lactic acid) polymer block; a poly(propylene glycol) polymer block; a poly(amino acid) polymer block; a poly(ester) polymer block; a poly (ε-caprolactone) polymer block; a poly(ethylene glycol) block, a poly(acrylic acid) block; a polylactide block; a polyester block; a polyamide block; a polyanhydride block; a polyurethane block; a polyimine block; a polyurea block; a polyacetal block; a polysaccharide block; and a polysiloxane block.

Suitable pharmaceutically-acceptable nonaqueous solvents include, but are not limited to, the following (as well as mixtures thereof):

(i) Alcohols (these include, for example, σ-glycerol formal, β-glycerol formal, 1, 3-butyleneglycol, aliphatic or aromatic alcohols having from 2 to about 30 carbons (e.g., methanol, ethanol, propanol, isopropanol, butanol, t-butanol, hexanol, octanol, amylene hydrate, benzyl alcohol, glycerin (glycerol), glycol, hexylene, glycol, tetrahydrofuranyl alcohol, cetyl alcohol, and stearyl alcohol), fatty acid esters of fatty alcohols (e.g., polyalkylene glycols, such as polypropylene glycol and polyethylene glycol), sorbitan, sucrose, and cholesterol);

(ii) Amides, which include, for example, dimethylacetamide (DMA), benzyl benzoate DMA, dimethylformamide, N-hydroxyethyO-lactamide, N, N-dimethylacetamide-amides, 2-pyrrolidinone, 1-methyl-2-pyrrolidinone, and polyvinylpyrrolidone;

(iii) Esters, which include, for example, acetate esters (e.g., monoacetin, diacetin, and triacetin), aliphatic and aromatic esters (e.g., ethyl caprylate or octanoate, alkyl oleate, benzyl benzoate, or benzyl acetate), dimethylsulfoxide (DMSO), esters of glycerin (e.g., mono, di, and tri-glyceryl citrates and tartrates), ethyl benzoate, ethyl acetate, ethyl carbonate, ethyl lactate, ethyl oleate, fatty acid esters of sorbitan, glyceryl monostearate, glyceride esters (e.g., mono, di, or triglycerides), fatty acid esters (e.g., isopropyl myristate), fatty acid derived PEG esters (e.g., PEG-hydroxyoleate and PEG-hydroxystearate), N-methyl pyrrolidinone, pluronic 60, polyoxyethylene sorbitol oleic polyesters (e.g., poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate), polyoxyethylene sorbitan esters (e.g., polyoxyethylene-sorbitan monooleate, polyoxyethylene-sorbitan monopalmitate, polyoxyethylene-sorbitan monolaurate, polyoxyethylene-sorbitan monostearate, and POLYSORBATE 20, 40, 60, and 80 (from ICI Americas, Wilmington, Del.)), polyvinylpyrrolidone, alkyleneoxy modified fatty acid esters (e.g., polyoxyl 40 hydrogenated castor oil and polyoxyethylated castor oils, such as CREMOPHOR EL solution or CREMOPHOR RH 40 solution), saccharide fatty acid esters (i.e., the condensation product of a monosaccharide (e.g., pentoses, such as, ribose, ribulose, arabinose, xylose, lyxose, and xylulose; hexoses, such as glucose, fructose, galactose, mannose, and sorbose; trioses; tetroses; heptoses; and octoses), disaccharide (e.g., sucrose, maltose, lactose, and trehalose), oligosaccharide, or a mixture thereof with one or more C₄-C₂₂ fatty acids (e.g., saturated fatty acids, such as caprylic acid, capric acid, lauric acid, myristic acid, palmitic acid, and stearic acid; and unsaturated fatty acids, such as palmitoleic acid, oleic acid, elaidic acid, erucic acid, and linoleic acid), and steroidal esters;

(iv) Ethers, for example, alkyl, aryl, and cyclic ethers having from 2 to about 30 carbons. Examples include diethyl ether, tetrahydrofuran, dimethyl isosorbide, diethylene glycol monoethyl ether), and glycofurol (tetrahydrofurofuranyl alcohol polyethylene glycol ether);

(v) Ketones which typically have from about 3 to about 30 carbons. Examples include acetone, methyl ethyl ketone, and methyl isobutyl ketone;

(vi) Hydrocarbons which are typically aliphatic, cycloaliphatic, or aromatic hydrocarbons having from about 4 to about 30 carbons. Examples include benzene, cyclohexane, dichloromethane, dioxolanes, hexane, n-decane, n-dodecane, n-hexane, sulfolane, tetramethylenesulfone, tetramethylenesulfoxide, toluene, dimethylsulfoxide (DMSO); and tetramethylene sulfoxide;

(vii) Oils which include, for example, oils of mineral, vegetable, animal, essential, or synthetic origin. These include: mineral oils, such as aliphatic and wax-based hydrocarbons, aromatic hydrocarbons, mixed aliphatic and aromatic based hydrocarbons, and refined paraffin oil; vegetable oils, such as linseed, tung, safflower, soybean, castor, cottonseed, groundnut, rapeseed, coconut, palm, olive, corn, corn germ, sesame, persic, and peanut oil; glycerides, such as mono-, di-, and triglycerides; animal oils, such as fish, marine, sperm, cod-liver, haliver, squalene, squalane, and shark liver oil; oleic oils; and polyoxyethylated castor oil;

(viii) Alkyl, alkenyl, or aryl halides which include, for example, alkyl or aryl halides having from 1 to about 30 carbons and one or more halogen substituents. Examples include: methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (SOLUTOL HS-15, from BASF, Ludwigshafen, Germany); polyoxyethylene glycerol; sodium laurate; sodium oleate; and sorbitan monooleate.

Other pharmaceutically acceptable solvents for use in the invention are well known to those of ordinary skill in the art. General discussion relating to such solvents can be found in, for example, The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics 3d ed., (G. Banker et. al., eds., Marcel Dekker, Inc., New York, N.Y. (1995)), The Pharmacological Basis of Therapeutics, (Goodman & Gilman, McGraw Hill Publishing), Pharmaceutical Dosage Forms, (H. Lieberman et. al., eds., Marcel Dekker, Inc., New York, N.Y. (1980)), Remington's Pharmaceutical Sciences, 19th ed., (A. Gennaro, ed., Mack Publishing, Easton, Pa., (1995)), The United States Pharmacopeia 24, The National Formulary 19, (National Publishing, Philadelphia, Pa. (2000)); Spiegel, A. J., et al., “Use of Nonaqueous Solvents in Parenteral Products,” J. Pharma. Sciences, Vol. 52, No. 10, pp. 917-927 (1963).

Solvents useful in the invention include, but are not limited to, those known to stabilize present compounds or pharmaceutically acceptable salts thereof. These can include, for example, oils rich in triglycerides, such as safflower oil, soybean oil, and mixtures thereof; and alkyleneoxy-modified fatty acid esters, such as polyoxyl 40 hydrogenated castor oil and polyoxyethylated castor oils (e.g., CREMOPHOR EL solution or CREMOPHOR RH 40 solution). Commercially available triglycerides include INTRALIPID emulsified soybean oil (Kabi-Pharmacia Inc., Stockholm, Sweden), NUTRALIPID emulsion (McGaw, Irvine, Calif.), LIPOSYN II 20% emulsion (a 20% fat emulsion solution containing 100 mg safflower oil, 100 mg soybean oil, 12 mg egg phosphatides, and 25 mg glycerin per ml of solution; Abbott Laboratories, Chicago, Ill.), LIPOSYN III 2% emulsion (a 2% fat emulsion solution containing 100 mg safflower oil, 100 mg soybean oil, 12 mg egg phosphatides, and 25 mg glycerin per ml of solution; Abbott Laboratories, Chicago, Ill.), natural or synthetic glycerol derivatives containing the docosahexaenoyl group at levels of from about 25 to about 100% (by weight based on the total fatty acid content) (DHASCO from Martek Biosciences Corp., Columbia, Md.; DHA MAGURO from Daito Enterprises, Los Angeles, Calif.; SOYACAL; and TRAVEMULSION). Ethanol in particular is a useful solvent for dissolving a compound or pharmaceutically acceptable salt thereof to form solutions, emulsions, and the like.

Additional components can be included in the compositions of this invention for various purposes generally known in the pharmaceutical industry. These components tend to impart properties that, for example, enhance retention of the present compounds or salt thereof at the site of administration, protect the stability of the composition, control the pH, and facilitate processing of the compound or salt thereof into pharmaceutical formulations, and the like. Specific examples of such components include cryoprotective agents; agents for preventing reprecipitation of the compound or salt surface; active, wetting, or emulsifying agents (e.g., lecithin, polysorbate-80, TWEEN 80, pluronic 60, and polyoxyethylene stearate); preservatives (e.g., ethyl-p-hydroxybenzoate); microbial preservatives (e.g., benzyl alcohol, phenol, m-cresol, chlorobutanol, sorbic acid, thimerosal, and paraben); agents for adjusting pH or buffering agents (e.g., acids, bases, sodium acetate, sorbitan monolaurate, etc.); agents for adjusting osmolarity (e.g., glycerin); thickeners (e.g., aluminum monostearate, stearic acid, cetyl alcohol, stearyl alcohol, guar gum, methyl cellulose, hydroxypropylcellulose, tristearin, cetyl wax esters, polyethylene glycol, etc.); colorants; dyes; flow aids; non-volatile silicones (e.g., cyclomethicone); clays (e.g., bentonites); adhesives; bulking agents; flavorings; sweeteners; adsorbents; fillers (e.g., sugars such as lactose, sucrose, mannitol, sorbitol, cellulose, calcium phosphate, etc.); diluents (e.g., water, saline, electrolyte solutions, etc.); binders (e.g., gelatin; gum tragacanth; methyl cellulose; hydroxypropyl methylcellulose; sodium carboxymethyl cellulose; polyvinylpyrrolidone; sugars; polymers; acacia; starches, such as maize starch, wheat starch, rice starch, and potato starch; etc.); disintegrating agents (e.g., starches, such as maize starch, wheat starch, rice starch, potato starch, and carboxymethyl starch; cross-linked polyvinyl pyrrolidone; agar; alginic acid or a salt thereof, such as sodium alginate; croscarmellose sodium; crospovidone; etc); lubricants (e.g., silica; talc; stearic acid and salts thereof, such as magnesium stearate; polyethylene glycol; etc.); coating agents (e.g., concentrated sugar solutions including gum arabic, talc, polyvinyl pyrrolidone, carbopol gel, polyethylene glycol, titanium dioxide, etc.); and antioxidants (e.g., sodium metabisulfite, sodium bisulfite, sodium sulfite, dextrose, phenols, thiophenols, etc.).

Techniques and compositions for making parenteral dosage forms are generally known in the art. Formulations for parenteral administration can be prepared from one or more sterile powders and/or granules having a compound or salt of this invention and one or more of the carriers or diluents mentioned for use in the formulations for oral administration. The powder or granule typically is added to an appropriate volume of a solvent (typically while agitating (e.g., stirring) the solvent) that is capable of dissolving the powder or granule. Particular solvents useful in the invention include, for example, water, polyethylene glycol, propylene glycol, ethanol, corn oil, cottonseed oil, peanut oil, sesame oil, benzyl alcohol, sodium chloride, and/or various buffers.

Emulsions for parenteral administration can be prepared by, for example, dissolving a compound or salt of this invention in any pharmaceutically acceptable solvent capable of dissolving the compound to form a solution; and adding an appropriate volume of a carrier to the solution while stirring to form the emulsion. Solutions for parenteral administration can be prepared by, for example, dissolving a compound or salt of this invention in any pharmaceutically acceptable solvent capable of dissolving the compound to form a solution; and adding an appropriate volume of a carrier to the solution while stirring to form the solution.

Suppositories for rectal administration can be prepared by, for example, mixing the drug with a suitable nonirritating excipient that is solid at ordinary temperatures, but liquid at the rectal temperature and will therefore melt in the rectum to release the drug. Suitable excipients include, for example, cocoa butter; synthetic mono-, di-, or triglycerides; fatty acids; and/or polyethylene glycols.

Every formulation or combination of components described or exemplified herein can be used to practice the invention, unless otherwise stated.

(i) Binding Agents: Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, the materials sold as AVICEL-PH-101, AVICEL-PH-103 and AVICEL-PH-105 (available from FMC Corporation, American Viscose Division, Avicel Sales, Marcus Hook, Pa., USA). An exemplary suitable binder is a mixture of microcrystalline cellulose and sodium carboxymethyl cellulose sold as AVICEL RC-581 by FMC Corporation.

(ii) Fillers: Fillers include, but are not limited to, talc, calcium carbonate (e.g., granules or powder), lactose, microcrystalline cellulose, powdered cellulose, dextrates, kaolin, mannitol, silicic acid, sorbitol, starch, pre-gelatinized starch, and mixtures thereof.

(iii) Lubricants: Lubricants include, but are not limited to, calcium stearate, magnesium stearate, mineral oil, electromagnetic radiation mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a syloid silica gel (AEROSIL 200, manufactured by W.R. Grace Co. of Baltimore, Md., USA), a coagulated aerosol of synthetic silica (marketed by Deaussa Co. of Plano, Tex., USA), CAB-O-SIL (a pyrogenic silicon dioxide product sold by Cabot Co. of Boston, Mass., USA), and mixtures thereof.

(iv) Disintegrants: Disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Tablets or capsules can optionally be coated by methods well known in the art. If binders and/or fillers are used with a compound/bioconjugate of the invention, they are typically formulated as about 50 to about 99 weight percent of the compound/bioconjugate. In one aspect, about 0.5 to about 15 weight percent of disintegrant, and particularly about 1 to about 5 weight percent of disintegrant, can be used in combination with the compound. A lubricant can optionally be added, typically in an amount of less than about 1 weight percent of the compound/bioconjugate. Techniques and pharmaceutically acceptable additives for making solid oral dosage forms are described in Marshall, SOLID ORAL DOSAGE FORMS, Modern Pharmaceutics (Banker and Rhodes, Eds.), 7:359-427 (1979). Other formulations are known in the art.

Liquid preparations for oral administration can take the form of solutions, syrups or suspensions. Alternatively, the liquid preparations can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations can also contain buffer salts, flavoring, coloring, perfuming and sweetening agents as appropriate. Preparations for oral administration can also be formulated to achieve controlled release of the compound/bioconjugate. Oral formulations preferably contain 10% to 95% compound/bioconjugate. In addition, a compound/bioconjugate of the present invention can be formulated for buccal administration in the form of tablets or lozenges formulated in a conventional manner. Other methods of oral delivery of compounds/bioconjugates of the invention will be known to the skilled artisan and are within the scope of the invention.

Formulation 1: Hard gelatin capsules prepared using the following:

TABLE F1

| Ingredients | (mg/capsule) |
|---|---|
| Active Ingredient | 250.0 |
| Starch | 305.0 |
| Magnesium stearate | 5.0 |

The ingredients are mixed and filled into hard gelatin capsules in 560 mg quantities.

Formulation 2: A tablet formula is prepared using the following ingredients:

TABLE F2

| Ingredients | (mg/tablet) |
|---|---|
| Active Ingredient | 250.0 |
| Cellulose, microcrystalline | 400.0 |
| Colloidal silicon dioxide | 10.0 |
| Stearic acid | 5.0 |

The components are blended and compressed to form tablets, each 665 mg.

Formulation 3: A dry powder inhaler formulation is prepared containing the following components:

TABLE F3

| Ingredients | Weight % |
|---|---|
| Active ingredient | 5 |
| Lactose | 95 |

The active ingredient is mixed with the lactose and the mixture is added to a dry powder inhaling appliance.

Formulation 4: Tablets, each containing 60 mg of active ingredient, are prepared as follows:

TABLE F4

| Ingredients | Milligrams |
|---|---|
| Active ingredient | 60.0 |
| Starch | 45.0 |
| Microcrystalline cellulose | 35.0 |
| Polyvinylpyrrolidone | 4.0 |
| (as 10% solution in water) | |
| Sodium carboxymethyl starch | 4.5 |
| Magnesium stearate | 0.5 |
| Talc | 1.0 |
| Total | 150.0 |

The active ingredient, starch and cellulose are passed through a No. 20 mesh U.S. sieve and mixed thoroughly. The solution of polyvinylpyrrolidone is mixed with the resultant powders which are then passed through a 16 mesh U.S. sieve. The granules as produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

Formulation 5: Capsules, each containing 80 mg of active ingredient are made as follows:

TABLE F5

| Ingredients | Milligrams |
|---|---|
| Active ingredient | 80.0 |
| Starch | 109.0 |
| Magnesium stearate | 1.0 |
| Total | 190.0 |

The active ingredient, cellulose, starch, and magnesium stearate are blended, passed through a No. 20 mesh U.S. sieve, and filled into hard gelatin capsules in 190 mg quantities.

Formulation 6: Suppositories, each containing 225 mg of active ingredient, are made as follows:

TABLE F6

| Ingredients | Milligrams |
|---|---|
| Active Ingredient | 225 |
| Saturated fatty acid glycerides to | 2000 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Formulation 7: Suspensions, each containing 50 mg of active ingredient per 5.0 ml dose are made as follows:

TABLE F7

| Ingredients | Milligrams | |
|---|---|---|
| Active ingredient | 50.0 | mg |
| Xanthan gum | 4.0 | mg |
| Sodium carboxymethyl cellulose | (11%) | |
| Microcrystalline cellulose | (89%) 50.0 | mg |
| Sucrose | 1.75 | g |
| Sodium benzoate | 10.0 | mg |
| Flavor | q.v. | |
| Color | q.v. | |
| Purified water to | 5.0 | ml |

The active ingredient, sucrose and xantham gum are blended, passed through a No. 10 mesh U.S. sieve, and mixed with a previously made solution of the microcrystalline cellulose and sodium carboxymethyl cellulose in water. The sodium benzoate, flavor, and color are diluted with some of the water and added with stirring. Sufficient water is then added to produce the required volume.

Formulation 8: Capsules, each containing 150 mg of active ingredient, are made as follows:

TABLE F8

| Ingredients | Milligrams |
|---|---|
| Active ingredient | 150.0 |
| Starch | 407.0 |
| Magnesium stearate | 3.0 |
| Total | 560.0 |

The active ingredient, cellulose, starch, and magnesium stearate are blended, passed through a No. 20 mesh U.S. sieve, and filled into hard gelatin capsules in 560 mg quantities.

Kits: Various embodiments of the present invention include kits. Such kits can include a compound/bioconjugate of the present invention, optionally one or more ingredients for preparing a pharmaceutically acceptable formulation of the compound/bioconjugate, and instructions for use (e.g., administration). When supplied as a kit, different components of a compound/bioconjugate formulation can be packaged in separate containers and admixed immediately before use. Such packaging of the components separately can, if desired, be presented in a pack or dispenser device which can contain one or more unit dosage forms containing the compound/bioconjugate. The pack can, for example, comprise metal or plastic foil such as a blister pack. Such packaging of the components separately can also, in certain instances, permit long-term storage without losing activity of the components. In addition, if more than one route of administration is intended or more than one schedule for administration is intended, the different components can be packaged separately and not mixed prior to use. In various embodiments, the different components can be packaged in one combination for administration together.

It is further contemplated that the compounds and salts of this invention can be used in the form of a kit that is suitable for use in performing the methods described herein, packaged in a container. The kit can contain the compound or compounds and, optionally, appropriate diluents, devices or device components suitable for administration and instructions for use in accordance with the methods of the invention. The devices can include parenteral injection devices, such as syringes or transdermal patch or the like. Device components can include cartridges for use in injection devices and the like. In one aspect, the kit includes a first dosage form including a compound or salt of this invention and a second dosage form including another active ingredient in quantities sufficient to carry out the methods of the invention. The first dosage form and the second dosage form together can include a therapeutically effective amount of the compounds for treating the targeted condition(s).

In certain embodiments, kits can be supplied with instructional materials. Instructions can be printed on paper or other substrate, and/or can be supplied as an electronic-readable medium, such as a floppy disc, mini-CD-ROM, CD-ROM, DVD-ROM, Zip disc, videotape, audio tape, and the like. Detailed instructions cannot be physically associated with the kit; instead, a user can be directed to an Internet web site specified by the manufacturer or distributor of the kit, or supplied as electronic mail.

If desired, the emulsions or solutions described above for oral or parenteral administration can be packaged in IV bags, vials, or other conventional containers in concentrated form, and then diluted with a pharmaceutically acceptable liquid (e.g., saline) to form an acceptable compound concentration before use.

Kits can include reagents in separate containers such as, for example, sterile water or saline to be added to a lyophilized active component packaged separately. For example, sealed glass ampules can contain lyophilized superoxide dismutase mimetics and in a separate ampule, sterile water, sterile saline or sterile each of which has been packaged under a neutral non-reacting gas, such as nitrogen. Ampules can consist of any suitable material, such as glass, organic polymers, such as polycarbonate, polystyrene, ceramic, metal or any other material typically employed to hold reagents. Other examples of suitable containers include bottles that can be fabricated from similar substances as ampules, and envelopes that can consist of foil-lined interiors, such as aluminum or an alloy. Other containers include test tubes, vials, flasks, bottles, syringes, and the like. Containers can have a sterile access port, such as a bottle having a stopper that can be pierced by a hypodermic injection needle. Other containers can have two compartments that are separated by a readily removable membrane that upon removal permits the components to mix. Removable membranes can be glass, plastic, rubber, and the like.

Example 4: In vitro efficacy and mite lifetimes: Relative efficacy of various formulations are evaluated with a demodex survival time experiments. FIG. 3 tabulates demodex survival time, expressed in terms of LT50 (time at which 50% of demodex mites are killed) and/or average minutes to death for various active agents. The compounds having an LT50 labeled “no activity” reflects an activity that is not significantly different from control (e.g., water).

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference). For example, U.S. Provisional Application Nos. 61/859,572, filed Jul. 29, 2013, 61/861,072, filed Aug. 1, 2013 and 61/953,290 filed Mar. 14, 2014, U.S. nonprovisional application Ser. No. 14/444,748 filed Jul. 28, 2014 (published Mar. 26, 2015 as U.S. Pub. No. 2015/0086596A1), and PCT Application No. PCT/US14/48420 filed Jul. 28, 2014 (published Feb. 5, 2015 as Pub. No. WO 2015/017328); and PCT Pub. No. WO 2015/195928, each of which is hereby incorporated by reference in their entireties to the extent not inconsistent herewith.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

I claim:

1. A method of treating an individual having an autoimmune disease, the method comprising the step of administering to the individual chloroquine and/or hydroxychloroquine in a dosage sufficient to inactivate at least a portion of Demodex mites in or on the individual, wherein the inactivation results in attenuation or cessation of one or more symptoms associated with inflammatory and/or immune responses to the Demodex mites that causes one or more symptoms associated with the autoimmune disease in the individual.

2. The method of claim 1, wherein substantially all of the Demodex mites are inactivated.

3. The method of claim 1, wherein the administering is by topical, oral or intravenous administration of the chloroquine and/or hydroxychloroquine.

4. The method of claim 1, wherein said chloroquine and/or hydroxychloroquine is a miticide or insecticide.

5. The method of claim 1, wherein the inactivated Demodex mites comprise demodex brevis and/or demodex folliculorum mites from hair follicles, skin, eyes, eyelids, eyelashes, or meibomian glands of the individual.

6. The method of claim 1, wherein said chloroquine and/or hydroxychloroquine is topically applied and is provided in a formulation to efficiently transport an active ingredient of said chloroquine and/or hydroxychloroquine into the epidermis or a subdermal region of the individual.

7. The method of claim 6, wherein said chloroquine and/or hydroxychloroquine is applied to hair follicles, skin, eyes, eyelids, eyelashes, and/or meibomian glands of the individual.

8. The method claim 1, wherein said administering step is by orally-administering or topically-applying, and said inactivation kills at least a portion of said Demodex mites, or renders at least a portion of said Demodex mites unable to reproduce.

9. The method of claim 1, wherein said step of orally-administering or topically-applying said chloroquine and/or hydroxychloroquine kills and eliminates said mites, and optionally said mites are Demodex brevis and/or Demodex folliculorum mites.

10. The method of claim 1, wherein the autoimmune disease is one or more of lupus erythematosus, rheumatoid arthritis, juvenile idiopathic arthritis, systemic lupus, systemic sclerosis, juvenile dermatomyositis, adult dermatomyositis, Sjögren's syndrome or porphyria cutanea tarda.

11. The method of claim 1, wherein the autoimmune disease is one or more of systemic lupus erythematosus, rheumatoid arthritis, juvenile idiopathic arthritis, systemic lupus, systemic sclerosis, juvenile and adult dermatomyositis, Sjögren's syndrome, porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis.

12. The method of claim 1, wherein the autoimmune disease affects the epidermis of the individual.

13. The method of claim 1, wherein the autoimmune disease affects the epidermis, the lymphatic system, muscles, joint or internal organs of the individual.

14. The method of claim 1, wherein the chloroquine and/or hydroxychloroquine is topically-applied and is formulated in a carrier lotion, cream, soap, wash, shampoo or gel.

15. The method of claim 14, wherein the chloroquine and/or hydroxychloroquine in the topically-applied lotion, cream, soap, wash, shampoo or gel has a concentration of between 0.001% to 5% by weight.

16. The method of claim 14, wherein the chloroquine and/or hydroxychloroquine in the topically-applied lotion, cream, soap, wash, shampoo or gel has a concentration of between 0.01% to 1% by weight.

17. The method of claim 14, wherein the chloroquine and/or hydroxychloroquine in the topically-applied lotion, cream, soap, wash, shampoo or gel has a concentration that is a lowest effective concentration for killing Demodex mites.

18. The method of claim 14, wherein a dosage of the chloroquine and/or hydroxychloroquine in the topically-applied lotion, cream, soap, wash, shampoo or gel is less than 150 mg/kg of body mass or between 0.001 mg per kg of body mass and 50 mg/kg of body mass.

19. The method of claim 18, wherein a dosage of the chloroquine and/or hydroxychloroquine in the topically-applied lotion, cream, soap, wash, shampoo or gel is a lowest dose effective for killing the Demodex mites.

20. The method of claim 14, wherein the topically-applied chloroquine and/or hydroxychloroquine is encapsulated inside microliposomes before being formulated into the carrier lotion, cream, soap, wash, shampoo or gel.

21. The method of claim 14, wherein the topically-applied chloroquine and/or hydroxychloroquine is applied to hair follicles, skin, eyes, eyelids, eyelashes, or Meibomian Gland areas affected by the ophthalmological affliction.

22. The method of claim 1, wherein the chloroquine and/or hydroxychloroquine is orally or intravenously delivered and is provided at a concentration that is the lowest concentration effective for killing Demodex mites.

23. The method of claim 1, wherein the chloroquine and/or hydroxychloroquine is orally or intravenously delivered with a dose that is less than 150 mg/kg of body mass or between 0.001 mg per kg of body mass and 50 mg/kg of body mass.

24. The method of claim 2, wherein chloroquine and/or hydroxychloroquine is topically-applied to hair follicles, skin, eyes, eyelids, eyelashes, or meibomian gland areas of the body where Demodex brevis and/or Demodex folliculorum mites exist.

25. The method of claim 24, wherein the topically-applied chloroquine and/or hydroxychloroquine is applied to substantially all hair follicles, skin, eyes, eyelids, eyelashes, or meibomian gland areas of the individual.

26. The method of claim 1, further comprising a step of applying the chloroquine and/or hydroxychloroquine to an individual's clothing, linens or both clothing and linens.

27. The method of claim 1, further comprising a step of orally-administering or topically-applying the chloroquine and/or hydroxychloroquine to others having contact with the individual in a dosage sufficient to kill and eliminate Demodex brevis and/or Demodex folliculorum mites from hair follicles and/or skin of the others.

28. The method of claim 27, wherein the others are selected from the group consisting of household members, children, spouses, partners, family members and domestic pets.

29. The method of claim 28, wherein the topically-applied chloroquine and/or hydroxychloroquine is applied to the hair follicles and/or skin of the individual.

30. The method of claim 1, wherein the chloroquine and/or hydroxychloroquine is topically applied and penetrates an outer layer of the skin of the individual, thereby exposing the Demodex brevis and/or Demodex folliculorum mites present below the outer layer of the skin to the chloroquine and/or hydroxychloroquine.

31. The method of claim 30, wherein the topically-applied chloroquine and/or hydroxychloroquine penetrates to a subdermal region of the skin of the individual, thereby exposing the Demodex brevis and/or Demodex folliculorum mites present in the subdermal region of the skin to the chloroquine and/or hydroxychloroquine.

32. The method of claim 1, wherein the chloroquine and/or hydroxychloroquine has mite inactivation activity at any one or more of: hair follicles, skin, eyes, eyelids, eyelashes, or meibomian gland areas by application at least once and not more than twice daily for a period of two to six weeks.

33. The method of claim 32, wherein the chloroquine and/or hydroxychloroquine is applied to the skin or portions thereof, during a first application period, thereby killing and eliminating adult Demodex brevis and/or Demodex folliculorum mites from hair follicles, skin, eyes, eyelids, eyelashes, or meibomian glands.

34. The method of claim 33, wherein the chloroquine and/or hydroxychloroquine is further applied to hair follicles, skin, eyes, eyelids, eyelashes, or meibomian during a second application period, thereby killing and eliminating from the said hair follicles, skin, eyes, eyelids, eyelashes, or meibomian glands Demodex brevis and/or Demodex folliculorum mites that have matured from a larval form and/or an egg form present on and/or in the said hair follicles, skin, eyes, eyelids, eyelashes, or meibomian glands during the first application period.

35. The method of claim 34, wherein the applied chloroquine and/or hydroxychloroquine is further applied to skin areas during a third application period, thereby killing and eliminating from said hair follicles, skin, eyes, eyelids, eyelashes, or meibomian glands Demodex brevis and/or Demodex folliculorum mites that have matured from a larval form and/or an egg form present on and/or in the said hair follicles, skin, eyes, eyelids, eyelashes, or meibomian glands during the first application period and/or the second application period.

36. The method of claim 34, wherein the first application period and the second application period are separated by at least five but no more than ten days.

37. The method of claim 34, wherein the first application period and the second application period are separated by at least seven days.

38. The method of claim 34, wherein the first application period and the second application period are separated by a time sufficient to allow the larva form to mature into an adult form and/or to allow the egg form to mature into the adult form.

39. The method of claim 35, wherein the second application period and the third application period are separated by at least five but no more than ten days.

40. The method of any of claim 35, wherein the second application period and the third application period are separated by at least seven days.

41. The method of any of claim 35, wherein the second application period and the third application period are separated by a time sufficient to allow the larva form to mature into an adult form and/or to allow the egg form to mature into the adult form.

42. The method of claim 1, wherein the chloroquine and/or hydroxychloroquine is intravenously-administered, orally-administered or topically-applied in a continued intermittent regime sufficient for prophylactic control of Demodex mite population in hair follicles, skin, eyes, eyelids, eyelashes, or meibomian glands of the individual.

43. The method of claim 42, wherein the chloroquine and/or hydroxychloroquine is intravenously administered at an chloroquine and/or hydroxychloroquine dose of 150 mg per kg of body mass or less or between 0.01 mg per kg of body mass and 50 mg per kg of body mass.

44. The method of claim 42, wherein the chloroquine and/or hydroxychloroquine is orally administered.

45. The method of claim 44, wherein the orally-administered chloroquine and/or hydroxychloroquine is administered as an oral dose of the chloroquine and/or hydroxychloroquine of a lowest dose effective for killing the Demodex mites.

46. The method of claim 44, wherein the orally-administered chloroquine and/or hydroxychloroquine is administered as a daily dose of 10 mg per kg of body mass.

47. The method of claim 44, wherein the orally-administered chloroquine and/or hydroxychloroquine is administered as a daily dose of 7.5 mg per kg of body mass.

48. The method of claim 44, wherein the orally-administered chloroquine and/or hydroxychloroquine is administered as a three times per day dose of 5 mg per kg of body mass.

49. The method of claim 44, wherein the orally-administered chloroquine and/or hydroxychloroquine is repeated about two to four times with spacing of three to seven days between them.

50. The method of claim 43, wherein the intravenous-administered chloroquine and/or hydroxychloroquine is administered as an intravenous dose of the chloroquine and/or hydroxychloroquine of a lowest dose effective for killing the Demodex mites.

51. The method of claim 43, wherein the intravenous-administered chloroquine and/or hydroxychloroquine is administered as a daily dose of 10 mg per kg of body mass.

52. The method of claim 43, wherein the intravenous-administered chloroquine and/or hydroxychloroquine is administered as a daily dose of 7.5 mg per kg of body mass.

53. The method of claim 43, wherein the intravenous-administered chloroquine and/or hydroxychloroquine is administered as a three times per day dose of 5 mg per kg of body mass.

54. The method of claim 43, wherein the intravenous-administered chloroquine and/or hydroxychloroquine is repeated about two to four times with spacing of three to seven days between them.

55. The method of any of claims 1-54, wherein the orally-administered or intravenous-administered chloroquine and/or hydroxychloroquine is formulated as a prodrug or pharmaceutically acceptable salt.

56. The method of claim 1, wherein an immune and/or inflammatory pathway response to the mites results from a presence of one or more pathogens associated with the mites in the individual.

57. The method of claim 56, wherein the elimination of the Demodex brevis and/or Demodex folliculorum mites from the individual results in a reduction in population of the one or more pathogens in the individual.

58. The method of claim 56, wherein the one or more pathogens comprises one or more bacteria from the genus staphylococcus or from the genus bacillus.

59. The method of claim 58, wherein the one or more bacteria comprise bacillus oleronius bacteria.

60. The method of claim 58, wherein the one or more bacteria comprise Staphylococcus epidermidis bacteria.

61. The method of claim 56, wherein the one or more pathogens are present on the outside of the Demodex brevis and/or Demodex folliculorum mites.

62. The method of claim 56, wherein the one or more pathogens are present inside of the Demodex brevis and/or Demodex folliculorum mites.

63. The method of claim 56, wherein the one or more bacteria are present in a digestive system of the Demodex brevis and/or Demodex folliculorum mites.

64. A method of treating an autoimmune disease comprising a step of topically, intravenously or orally delivering to an individual having the autoimmune disease an active ingredient comprising an chloroquine and/or hydroxychloroquine in a dosage sufficient to inactivate Demodex mites from the individual, resulting in amelioration or cessation of the manifestations of immune and inflammatory responses to the mites that cause symptoms and signs of the autoimmune disease in the individual, wherein the active ingredient is applied or delivered to areas affected by the autoimmune disease and to areas not affected by the autoimmune disease.

65. The method of claim 64, wherein the Demodex mites are one or more of: Demodex aries, Demodex aurati, Demodex brevis, Demodex bovis, Demodex canis, Demodex caprae, Demodex caballi, Demodex cati, Demodex conicus, Demodex cornei, Demodex criceti, Demodex equi, Demodex folliculorum, Demodex foveolator, Demodex gapperi, Demodex gatoi, Demodex huttereri, Demodex injai, Demodex leucogasteri, Demodex microti, Demodex ovis, Demodex phyloides, Demodex ponderosus, Demodex vibrissae and Demodex zalophi.

66. The method of claim 64, wherein the Demodex mites are one or more of: Demodex brevis or Demodex folliculorum.

67. The method of claim 64, wherein the topically-applied active ingredient is applied to substantially all skin of the individual, thereby killing and eliminating the Demodex brevis and/or Demodex folliculorum mites from all skin of the individual.

68. The method of claim 64, wherein the active ingredient comprises a miticide or insecticide.

69. The method of claim 64, wherein the autoimmune disease comprises one or more of: systemic lupus erythematosus, rheumatoid arthritis, juvenile idiopathic arthritis, systemic lupus, systemic sclerosis, juvenile and adult dermatomyositis, Sjögren's syndrome, porphyria cutanea tarda, palindromic rheumatism, eosinophilic fasciitis, polymorphous light eruption, granuloma annulare, lichen planus, lupus panniculitis, discoid lupus, porphyria cutanea tarda, psoriatic arthritis, chronic ulcerative stomatitis, refractory chronic urticaria, sarcoidosis, frontal fibrosing alopecia, necrobiosis lipoidica, actinic reticuloid, actinic prurigo, epidermolysis bullosa, Kikuchi-Fujimoto disease, graft-versus-host disease, chronic erythema nodosum, morphea and systemic sclerosis, pemphigus vulgaris, pemphigus foliaceus and pemphigoid gestationis.

70. A method of treating an autoimmune affliction comprising a step of orally-administering, intravenously-administering or topically-applying to an individual having the autoimmune affliction chloroquine and/or hydroxychloroquine in a dosage sufficient to inactivate Demodex brevis and/or Demodex folliculorum mites from the individual resulting in amelioration or cessation of the manifestations of allergic and/or inflammatory responses to the mites that cause symptoms and signs of the autoimmune affliction in the individual.

71. The method of claim 70, wherein the mites are selected from the group consisting of: Demodex brevis; Demodex folliculorum; and Demodex brevis and Demodex folliculorum.

72. The method of claim 70, wherein said compound is hydroxychloroquine.

73. A method of alleviating in a subject symptoms associated with an autoimmune disease caused by Demodex organisms, the method comprising administering chloroquine and/or hydroxychloroquine to the subject having the autoimmune disease in a dosage sufficient to inactivate at least a portion of the Demodex organisms.

74. The method of claim 74, further comprising the step of identifying a subject in need of alleviating symptoms associated with the autoimmune disease caused by demodex organisms.

75. The method of claim 74, further comprising the step of monitoring whether the subject experiences symptom alleviations.

76. The method of claim 74, wherein the administration is topically, orally, or intravenously providing the chloroquine and/or hydroxychloroquine to the subject.

77. The method of claim 74, wherein the administration is a topical administration.

78. The method of claim 77, wherein the topical administration comprises applying the chloroquine and/or hydroxychloroquine to the face and hair follicles of the face and head.

79. The method of claim 77, wherein the topical administration comprises applying the chloroquine and/or hydroxychloroquine to substantially the entire body.

80. The method of claim 74, further comprising the step of sampling demodex levels in the patient after the administering step.

81. The method of claim 81, wherein the sampling step comprises one or more of: visualization of a skin surface, swabbing a skin surface, removing hair, a skin surface biopsy using an adhesive, a skin biopsy, or staining to visualize demodex such as by Löffler's alkaline methylene blue staining.

82. The method of claim 14, wherein the chloroquine and/or hydroxychloroquine in the topically-applied lotion, cream, soap, wash, shampoo or gel has a concentration of between 0.001 percent to 15 percent by weight.
